(19) United States
(12) Patent Application Publication
(10) Pub. No.: US 2007/0123778 A1
(43) Pub. Date: May 31, 2007

(54) Bladder measurement

(75) Inventor: Edward Kantorovich, Gan-Haim (IL)

Correspondence Address: WOLF, BLOCK, SCHORR & SOLIS-COHEN LLP, 250 PARK AVENUE, NEW YORK, NY, 10177, US

(73) Assignee: Volurine Israel Ltd., Jerusalem (IL)

(21) Appl. No.: 10/575,307

(22) PCT Filed:
(86) PCT No.: PCT/IL04/00940

(30) Foreign Application Priority Data
Oct. 13, 2003 (IL) 158379

(51) Int. Cl. A61B 8/00

(52) U.S. Cl. 600/437

(57) ABSTRACT

A measuring device, comprising, (a) an ultrasonic acoustic transceiver unit capable of sending and receiving acoustic signals into the body of a patient; (b) frequency modulating circuitry which drives said transceiver unit with a signal whose frequency varies with time; and (c) processing circuitry which extracts an indication of a distance, from at least one signal detected by said transceiver unit, said signal being a reflection of a transmission of said transceiver unit driven by said time varying frequency signal. Optionally, the indication is a distance between walls of a bladder.

### FIELD OF THE INVENTION

The present invention relates to measuring of parameters of a bladder, for example, absolute or relative distension.

### BACKGROUND OF THE INVENTION

Many people suffer from bladder volume related dysfunctions such as Over Active Bladder, elevated Post Void Residual Volumes associated with benign prostate hyperplesia, intermittent catheterization to enforce voiding or the use of artificial sphincters or other micturation controlling devices typically used to increase functional capacity, enuresis (uncontrolled voiding) of young children or elderly people who have lost bladder control or people that suffer from injury or a disability. Additionally, some people suffer from enuresis while sleeping or unconscious.

Many devices have been suggested to monitor a person's bladder in order to notify the person or a caretaker when the bladder is at a level that requires relief.

U.S. Pat. No. 4,926,871 to Ganguly et al., the disclosure of which is incorporated herein by reference, discloses a system for automatically determining the volume of the bladder using an ultrasound scanner that transmits a plurality of signals into the bladder and computes the volume from the received reflections.

U.S. Pat. No. 5,235,985 to McMorrow et al., the disclosure of which is incorporated herein by reference, discloses a system using multiple transducers to form a three dimensional image of the bladder and calculate the bladder volume.

U.S. Pat. No. 6,110,111 to Barnard, the disclosure of which is incorporated herein by reference, discloses a system which scans a bladder with multiple ultrasound signals and calculates a volume of the bladder.

U.S. Pat. No. 6,213,949 to Ganguly et al., the disclosure of which is incorporated herein by reference, discloses a system which transmits multiple scan signals into a bladder to determine cross-points with the front and back walls of the bladder. The system uses these points to determine an outline of the bladder and estimate the bladder volume.

It has apparently been suggested to scan a diameter of the bladder from the front wall to the back wall and estimate its volume assuming it to be a sphere or other simple geometric shapes. However there are several problems with this idea:

1. The bladder does not have a simple geometric shape, often what is measured as a diameter is actually only a chord.

2. The shape of the bladder expands non-uniformly since it is affected by neighbor organs.

3. Neighbor organs introduce noise and otherwise affect the returned signals, misleading devices in determining distention of the bladder.

4. Detecting the walls is not always trivial.

U.S. Pat. No. 5,058,591 to Companion et al., the disclosure of which is incorporated herein by reference, discloses a system that computes distention of the bladder in lower fill levels by comparing the timing and energy of an ultrasonic signal (e.g., a pulse) transmitted into the bladder and the resulting reflected signal. In higher fill levels, Companion suggests that the bladder moves, so distension is not a sufficient marker.

U.S. Pat. No. 6,579,247 to Abramovitch et al., the disclosure of which is incorporated herein by reference, discloses an inexpensive and possibly disposable device to determine the level of urine in the bladder. Abramovitch discloses using a low frequency sensor using audio waves 16 Hz-20 KHz or infrasound (less than 16 Hz), but not ultrasound, so as to minimize the power required by the device. Abramovitch measures phase changes.

WO 03/039343 to Roe, the disclosure of which is incorporated herein by reference, describes a method of urinary continence training using devices such as those described in the above patents.

### SUMMARY OF THE INVENTION

An aspect of some embodiments of the invention relates to measuring one or more parameters of a human urinary bladder using a frequency modulated ultrasonic signal. In an exemplary embodiment of the invention, the frequency of the ultrasonic signal follows a sweep function, so that different times of arrival are determined using different detection frequencies. Optionally, the sweep function is linear. In some embodiments of the invention, the sweep function is a symmetrical function, for example a sine wave. Alternatively, the sweep function is asymmetric, for example a saw tooth.

In an exemplary embodiment of the invention, the detected signals are used to determine a distance between near and far bladder walls. Optionally, this distance is used to estimate bladder fill level and/or other bladder parameters. In an exemplary embodiment of the invention, other bladder measurements and/or parameters are made in addition or instead of inter-wall distance, for example, bladder wall thickness or bladder wall compliance.

In an exemplary embodiment of the invention, a single transducer is used for transmission and for reception, so that the transducer itself heterodynes the received signal with the transmitted signal and a detector receives a signal representative of the difference in frequency.

A potential advantage of frequency sweeping is that a longer integration time can be provided, possibly on the order of milliseconds, as compared to time of flight based measurement techniques where short pulses are used. Optionally, the received signal is a continuous signal. A potential advantage of long integration time is that a lower voltage may be used to drive an ultrasonic transmitter. Optionally, the voltage is less than 15 Volts, as compared to 100 Volts and more for short pulse schemes. A potential advantage of frequency sweeping is that the received signal may be detected and processed at lower frequencies (e.g., after heterodyning) than the transmitted signal, for example, 30%, 20%, 10% thereof or less, for example 200 KHz or less.

A potential advantage of using a frequency sweeping based determination method is relative noise immunity and more accurate distance determination in an organ such as the bladder where a significant part of the organ does not reflect (e.g., the urine between the walls). In an exemplary embodiment of the invention, the amplitude difference between reflected signals from bladder walls and lack of reflection from urine is used to assist in signal detection.

A potential advantage of using sweeping signals is that signals from distances that are not of interest may be filtered out using a frequency filter. Optionally, a trade-off is provided between sweep rate, simplicity of processing and dimensional accuracy.

Optionally, signal processing methods for extracting some types of data are used. Optionally, once the distances to the walls are detected, these distances are used to set a signal processing “time of arrival window” for detecting and processing non-sweeping signals or for more detailed analysis of sweeping signals. Another potential advantage of a frequency sweeping signal is that providing a damper (used for short signals) may be avoided, possibly reducing unit cost and/or complexity. The use of short signal can require more complicated electronics to support pulse mode generation and detection.

In an exemplary embodiment of the invention, a bladder monitoring device is provided and used to alert (e.g., audio, visual and/or tactile) a user when his (or a patient's) bladder is full (e.g., above a threshold), or a fill level thereof. Optionally, the device is calibrated using a test micturation (urination) into a measuring cup. Optionally, the measuring cup measures and reports the volume, optionally directly to the device.

Alternatively or additionally to fill level alerts, a bladder monitoring device is optionally used for providing urodynamics parameters and/or other bladder physiological parameters, for example, for tracking the effects (including side effects) of drugs, tracking production of urine, tracking or diagnosis of bladder conditions, detecting and/or monitoring dehydration, detecting and/or monitoring bladder slow waves and/or for providing feedback, for example for urge incontinence, for bladder training and/or as a feedback for volume driven operation of artificial micturation control devices such as an artificial sphincter or a catheter used for intermittent catheterization. Optionally, the tracked parameters are determined using one or more of: distance between front and back walls, bladder wall thickness, wall attenuation, changes over time, past history, voiding rate and/or rate of filling of bladder. In an exemplary embodiment of the invention, one or more of these parameters is determined using a prior art bladder distention device, for example, reprogrammed to extract and/or process one or more of the above parameters.

In some embodiments of the invention, a single transducer is used to transmit and receive the signal. Alternatively, multiple displaced transducers are used to provide tolerance in deploying the device and/or movement of the bladder. In some embodiments of the invention, the bladder monitor device transmits a signal from each transducer in series and acts on the results that are clearest. Alternatively or additionally, the transducers can transmit simultaneously or in overlapping time bands. Signals from the various transducers are optionally differentiated by transmitting with different characteristics. For example, different frequencies and/or sweep functions and/or instantaneous frequencies (e.g., separated by a range corresponding to the distance of the far wall of the bladder) and/or sweep function repetition rates are used for different transducers. Alternatively or additionally, the beams of the transducers are formed so that they interfere less with each other.

An aspect of some embodiments of the invention relates to using an individual value of bladder distension to estimate a bladder fill level or volume. Optionally, the only measurement used to estimate fill level is a measure of distension, optionally a distance between near and far walls of the bladder.

An aspect of some embodiments of the invention relates to a calibration method for a bladder monitor device. In some embodiments of the invention, the bladder monitor device is operated in a calibration mode wherein bladder distension and user input is used to calibrate the correlation between the bladder distension size and bladder volume. In some embodiments of the invention, a functional relationship is assumed between the measured distance and volume. In some embodiments, an automated measuring device generates a signal indicative of a volume of micturation.

An aspect of some embodiments of the invention relates to a bladder monitor configured to correct measurements for off-center bladder position. In an exemplary embodiment of the invention, the configuration includes a sensor in a direction of off-setting of the bladder.

An aspect of some embodiments of the invention relates to using urine to assist in detection of a wall. In an exemplary embodiment of the invention, urine is assumed to lack any refection, therefore a signal detected in frequencies corresponding to the location of urine can be treated as noise and optionally used to set a noise trigger level.

An aspect of some embodiments of the invention relates to an acoustic pad having an adhesive layer for attachment to a body and/or an acoustic unit. Optionally, two adhesive layers are provided, one for attachment to the body and one for attachment to the unit. Optionally, the pad is molded into a shape which corresponds to a concave or other shape of the unit.

An aspect of some embodiments of the invention relates to using a bladder fill level as an indicator for bladder relief. In an exemplary embodiment of the invention, a person that uses manual bladder relief uses a fill level signal as an indicator for applying such relief instead of a time indication. Optionally, the time indication (e.g., every 2 hours) is used for indicating to the person that a fill level detection device should be used. In an exemplary embodiment of the invention, a bladder fill level is used over a considerable period of time, such as 2 days, one week, two weeks, a month or more, for at least 30%, 50% or more of the bladder relief events. In an exemplary embodiment of the invention, the fill level detection device is worn by the person. Optionally, the indication of filling is an alert provided to the user. Alternatively, it is applied manually when a fill level determination is desired.

An aspect of some embodiments of the invention relates to continuous measurement of bladder parameters. In an exemplary embodiment of the invention, one or more bladder parameters are sampled periodically, for example, every few seconds or minutes and changes in the parameter values are tracked. Exemplary parameters which may be tracked include wall thickness, volume, fill rate, urination rate and slow wave properties. Optionally, the parameter tracked is a discrete parameter, for example a urination event. Optionally, the details of the event are tracked with a relatively high sampling rate, for example every few seconds or every second or fraction of a second. Optionally, the tracked values are stored for later analysis, for example for diagnosis. Alternatively or additionally, the tracked parameters are used to decide on alert situations, for example detecting an abnormally high bladder fill rate, which may indicate an abnormal dose of diuretics.

An aspect of some embodiments of the invention relates to design of transducer parameters. In an exemplary embodiment of the invention, the transducer size and shape and operating frequency are selected so that the area of interest (e.g., the bladder) is within a Fresnel area of the ultrasonic beam. Optionally, the near field (e.g., typically about one transducer length) is before the area of interest and the far field (e.g., Fraunhofer area) is past the area of interest.

In an exemplary embodiment of the invention, the beam is designed to be focused (optionally by reason of being in a Fresnel area), so that the beam portion hitting a convex surface (e.g., outside surface of a front bladder wall) is converging and the beam hitting a concave surface (e.g., inside surface of a far bladder wall) is diverging.

There is also provided in accordance with an exemplary embodiment of the invention, a measuring device, comprising:

(a) an ultrasonic acoustic transceiver unit capable of sending and receiving acoustic signals into the body of a patient;

(b) frequency modulating circuitry which drives said transceiver unit with a signal whose frequency varies with time; and

(c) processing circuitry which extracts an indication of a distance, from at least one signal detected by said transceiver unit, said signal being a reflection of a transmission of said transceiver unit driven by said time varying frequency signal. Optionally, said signal is a sweeping frequency. Optionally, said sweeping is non-spatial.

In an exemplary embodiment of the invention, said indication comprises an indication of a distance between a near wall and a far wall of a bladder. Optionally, said processing circuitry estimates a fill level of said bladder from said indication. Optionally, the device comprises a memory having stored therein at least one calibration value used by said processing circuitry.

In an exemplary embodiment of the invention, the device comprises at least one user input control. Optionally, at least one of said at least one user input generates an indication that a bladder is full. Alternatively or additionally, at least one of said at least one user input generates an indication that a bladder is just emptied.

In an exemplary embodiment of the invention, at least one of said at least one user input is operative to receive a volume indication.

In an exemplary embodiment of the invention, said processing circuitry is operative to automatically detect a maximum fill level condition by tracking fill level over time. Alternatively or additionally, said processing circuitry is operative to estimate a residual fill volume.

In an exemplary embodiment of the invention, said indication of a distance comprises a bladder wall thickness.

In an exemplary embodiment of the invention, said processing circuitry is operative to estimate at least a bladder fill rate based on said indication.

In an exemplary embodiment of the invention, said time varying frequency comprises a saw-tooth linear sweep.

In an exemplary embodiment of the invention, said transceiver unit comprises a single ultrasonic element.

In an exemplary embodiment of the invention, said transceiver unit generates a converging beam focused at a distance between 30 and 50 mm.

In an exemplary embodiment of the invention, said transceiver unit generates a converging beam focused at a distance between 50 and 90 mm.

In an exemplary embodiment of the invention, said transceiver unit generates a beam having a Fresnel zone covering a range of possible values for said distance. Optionally, said range is between 20 and 200 mm.

In an exemplary embodiment of the invention, said transceiver unit comprises at least one transmitter and at least one separate receiver.

In an exemplary embodiment of the invention, said transceiver unit comprises at least two transmitters and wherein said processing circuitry selects between the signal received from the two transmitters, for extracting said indication. Optionally, said two transmitters comprises at least three transmitters arranged two in a line and one off of the line, and each configured to aim a beam in a same general direction, but not parallel to each other. Alternatively or additionally, said two transmitters transmit ultrasonic beams that are not parallel to each other.

In an exemplary embodiment of the invention, said unit is mounted on a concave surface.

In an exemplary embodiment of the invention, the device comprises a strap of a length suitable for placing around a trunk of a human for mounting said unit adjacent a urinary bladder.

In an exemplary embodiment of the invention, said device comprises an adhesively removable gel pad adapted for coupling said transducer unit to human skin.

In an exemplary embodiment of the invention, said device comprises a memory storing a history of extracted indications. Optionally, said device comprises a transmitter for transmission of data from said memory. Alternatively or additionally, said memory serves as a urination diary memory.

In an exemplary embodiment of the invention, the device comprises an alert generator which generates an alert based on said indication. Optionally, said alert is tactile.

In an exemplary embodiment of the invention, said acoustic transceiver unit transmits signals at between 200 KHz and 2000 KHz.

In an exemplary embodiment of the invention, said time varying frequency varies from a minimum frequency to a maximum frequency over a time period greater than a travel time of signals at said frequency from a near wall to a far wall of a distended adult bladder. Optionally, said time period is at least 10 times the travel time. Optionally, said time period is at least 20 times the travel time.

In an exemplary embodiment of the invention, said processing circuitry is adapted for processing only frequencies lower than 200 KHz.

In an exemplary embodiment of the invention, said processing circuitry is adapted for processing only frequencies lower than 150 KHz.

In an exemplary embodiment of the invention, said transceiver comprises a piezoelectric material excited with less than 13V.

In an exemplary embodiment of the invention, said transceiver comprises a piezoelectric material excited with less than 5V.

In an exemplary embodiment of the invention, said transceiver comprises at least one transducer which acts as both a transmitter and a receiver and which heterodynes a received signal with a transmitted signal.

In an exemplary embodiment of the invention, said device comprises a frequency filter which drops frequencies corresponding to distances not of interest.

In an exemplary embodiment of the invention, said processing circuitry accumulate the contribution of at least 1 millisecond of received signals for said extracting.

There is also provided in accordance with an exemplary embodiment of the invention, a method of measuring a parameter of a bladder, comprising:

(a) transmitting a time-varying frequency modulated ultrasonic signal at a bladder;

(b) receiving a reflection of said signal from at least a portion of said bladder; and

(c) extracting at least an indication of a distance from a frequency of said reflection.

Optionally, transmitting a time-varying signal comprises transmitting a plurality of time-varying signals from spatially separated transducers. Optionally, the method comprises selecting a best reflection.

In an exemplary embodiment of the invention, said indication comprises a distance to a far wall of said bladder.

In an exemplary embodiment of the invention, said indication comprises a wall thickness.

In an exemplary embodiment of the invention, said indication comprises a distance between a near wall and a far wall of said bladder. Optionally, the method comprises converting said indication into an estimation of a fill level of said bladder. Optionally, said fill level comprises a fill volume. Alternatively or additionally, the method comprises generating an alert to a user responsive to said fill level. Alternatively or additionally, the method comprises calibrating said indication to a fill level. Optionally, calibrating comprises receiving a filling indicator from a user. Optionally, the method comprises measuring an output of urine in a measuring device outside the body. Optionally, said device generates an electronic signal reflecting a urine volume.

In an exemplary embodiment of the invention, calibrating comprises automatically tracking filling and emptying behavior of said bladder.

In an exemplary embodiment of the invention, said indication comprises a thickness of a bladder wall. Optionally, the method comprises extracting an indication of bladder slow waves in said bladder.

In an exemplary embodiment of the invention, the method comprises extracting a bladder fill rate from said indication.

In an exemplary embodiment of the invention, the method comprises extracting a urination rate from said indication.

In an exemplary embodiment of the invention, the method comprises extracting a residual urine volume from said indication.

In an exemplary embodiment of the invention, the method comprises tracking and storing said indication over time.

There is also provided in accordance with an exemplary embodiment of the invention, a method of calibrating a bladder urine fill detector device, comprising:

(a) measuring a volume of urine from a urination event;

(b) measuring a physical parameter of a bladder in association with said event; and

(c) storing a correspondence between said volume and said physical parameter in said detector device. Optionally, measuring comprises measuring a distension of said bladder using a frequency sweeping method.

In an exemplary embodiment of the invention, said parameter comprises a volume of urine. Optionally, said volume is measured using a sensor and automatically provided to said detector device.

In an exemplary embodiment of the invention, said parameter comprises a bladder wall thickness.

There is also provided in accordance with an exemplary embodiment of the invention, a method of measuring bladder distension, comprising:

(a) sending a measurement signal to an off-center bladder to measure a one dimensional geometrical parameter of said bladder; and

(b) estimating a distension of said bladder from said measured parameter.

There is also provided in accordance with an exemplary embodiment of the invention, a method of detecting a wall of a bladder, comprising:

(a) detecting a level of acoustic signal ostensibly from urine; and

(b) searching for a signal from a bladder wall to be greater in amplitude than the detected level.

There is also provided in accordance with an exemplary embodiment of the invention, a method of estimating a bladder fill level above 70% thereof, comprising:

(a) transmitting an acoustic signal to the bladder;

(b) determining an indication of a distension of said bladder in one dimension; and

(c) determining a fill level of said bladder to be above 70%, from said indication, using at least one personalized calibration value. Optionally, said acoustic signal comprises a frequency swept scalar signal.

There is also provided in accordance with an exemplary embodiment of the invention, a method of selecting device parameters, comprising:

(a) providing a device design for use in a body geometry and having at least one of a selectable acoustic transducer geometry and an operating frequency; and

(b) selecting at least one of said operating frequency and said body geometry such that said body geometry lies within a Fresnel zone of said transducer operating at said frequency.

In an exemplary embodiment of the invention, a method of bladder device calibration comprises estimating a pressure in said bladder using a catheter and determining a correspondence between said indication of distance and said pressure.

In an exemplary embodiment of the invention, in a bladder measuring device processing circuitry is operative to estimate at least one urodynamic parameter based on said indication.

There is also provided in accordance with an exemplary embodiment of the invention, a method of void selecting, comprising:

(a) detecting a fill level of bladder using a sensor from outside the body;

(b) deciding if the fill level requires voiding; and

(c) activating a manually activated bladder voiding mechanism. Optionally, said voiding mechanism comprises intermittent catheterization. Optionally, said detecting comprises detecting using a sweeping frequency modulated scalar acoustic signal.

### BRIEF DESCRIPTION OF FIGURES

Particular exemplary embodiments of the invention will be described with reference to the following description of embodiments in conjunction with the figures, wherein identical structures, elements or parts which appear in more than one figure are generally labeled with a same or similar number in all the figures in which they appear, in which:

FIG. 1A is a schematic diagram illustrating the principle of operation of a bladder filling monitor, in accordance with an exemplary embodiment of the invention;

FIG. 1B is a graph showing a measured relationship between bladder inter-wall distance and bladder volume, in accordance with an exemplary embodiment of the invention;

FIG. 2A is a graph illustrating using a frequency sweeping method for determining distance, in accordance with an exemplary embodiment of the invention;

FIG. 2B is a graph illustrating an exemplary received signal, processed and converted to the frequency domain, in accordance with an exemplary embodiment of the invention;

FIG. 3 is a flowchart of a method of bladder filling monitoring in accordance with an exemplary embodiment of the invention;

FIGS. 4A and 4B are graphs showing actually measured signals, in an experiment in accordance with an exemplary embodiment of the invention;

FIG. 5 is a flowchart of a method for calibration, in accordance with an exemplary embodiment of the invention;

FIG. 6 is a schematic block diagram of a bladder monitor in accordance with an exemplary embodiment of the invention;

FIG. 7A is a schematic illustration of a multiple-transducer layout for a bladder monitor, in accordance with an exemplary embodiment of the invention;

FIG. 7B is a side cross-sectional view of bladder monitor of FIG. 7A, in accordance with an exemplary embodiment of the invention;

FIG. 8 is a graph showing measurements of a bladder, in accordance with an exemplary embodiment of the invention;

FIGS. 9A-9C are graphs showing results of measurements during urination, in accordance with an exemplary embodiment of the invention; and

FIG. 10 is a flowchart of a method of controlled bladder relief in accordance with an exemplary embodiment of the invention.

### DETAILED DESCRIPTION OF EMBODIMENTS

General Overview

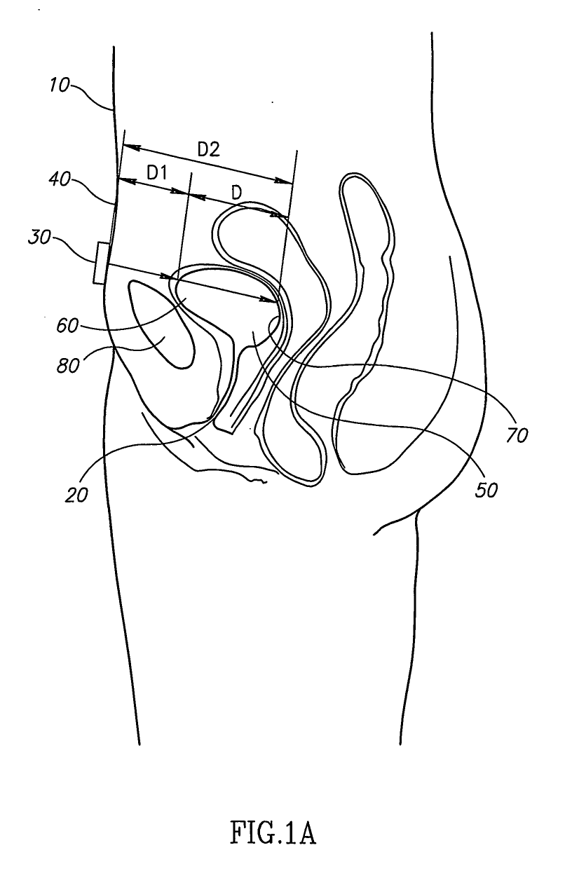

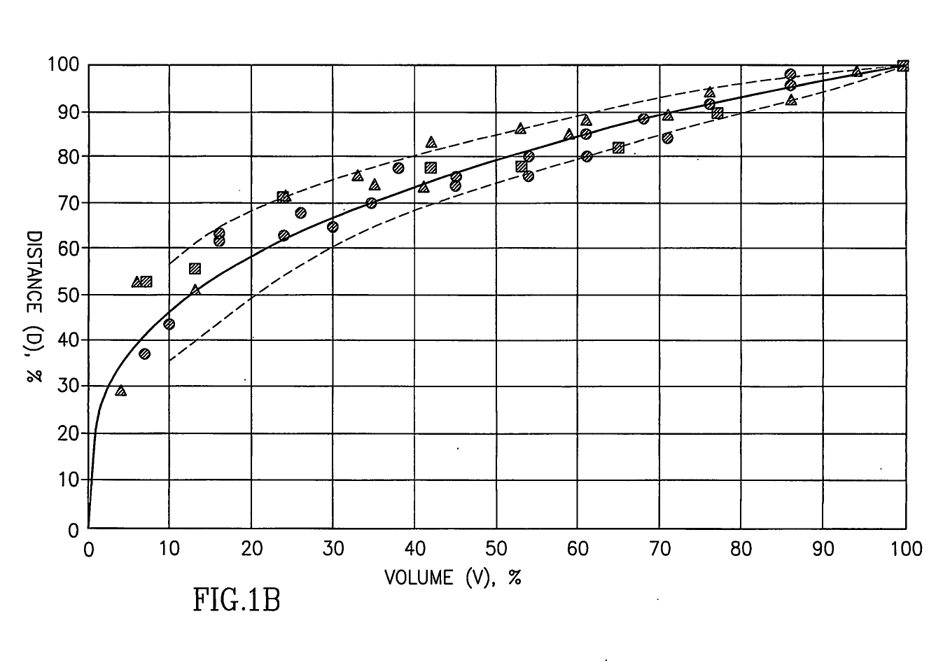

FIG. 1 is a schematic diagram illustrating the principle of operation of a bladder filling monitor, according to an exemplary embodiment of the invention. In FIG. 1 a bladder monitoring device 30 is positioned on the abdomen 40 of a user 10 such that an ultrasonic signal can be transmitted from the device toward the user's urinary bladder 50. Optionally bladder monitor device 30 is positioned above a pubic bone 80, which would otherwise block the signals. A urethra 20 through which an intermittent catheterization may be performed, in accordance with some embodiments of the invention, is also shown.

When a signal is transmitted toward a front wall 60 of bladder 50, some of the signal is reflected back to device 30. In an exemplary embodiment of the invention, some of the signal penetrates front bladder wall 60 and continues through the urine in the bladder to a back wall 70 of bladder 50. At back wall 70 some of the signal penetrates the wall and some of the signal is reflected back to device 30.

Generally, the first two significant reflected signals represent the reflection from front bladder wall 60 and from back bladder wall 70, respectively. The signal from back bladder wall 70 is typically the strongest signal received, due to the concavity of the bladder wall (when distorted). By analyzing the time difference of the signals, as will be described below, distances D1 (to front bladder wall 60) and D2 (to back bladder wall 70) can be determined. An inter-wall distance (distention) D, can be calculated as D=D2−D1. Alternatively, the distance may be determined directly without first determining D1 and D2. In some operating modes, device 30 is used to measure other parameters of the bladder, for example, wall thickness, slow waves, fill rate and/or emptying rate.

As determined by experimentation, front bladder wall 60 is generally immobile and does not move more than 3-4 mm while bladder 50 is filling up. Typically, distance D1 is between 20 mm and 50 mm in a person. In contrast to the relative stability of D1, D2 changes so that distention distance D typically expands from 30 mm to 10 mm while the volume of bladder 50 increases from 25% to 100% of a maximum volume.

Bladder 50 typically does not have a simple spherical shape and does not expand uniformly in all directions. FIG. 1B is a graph showing a relationship between bladder inter-wall distance and bladder volume, as measured in accordance with an exemplary embodiment of the invention, for several different people. The lines indicate an expected theoretical inverse cubic relationship and deviations therefrom. The measurements are normalized to “maximum volume”. As shown in FIG. 1B, the relationship between the actual bladder volume (as measured by measuring urine volume) and bladder distention is relatively similar for different people, especially once the bladder is at least 20% fill. Also, while the relationship is generally inverse-cubic, at higher fill levels, the relationship is approximately linear. In an exemplary embodiment of the invention, the shown linear or near-linear relationship is used for estimating bladder volume from distention measurements. Alternatively, the curve is used, for example as in function form (e.g., approximation function such as a polynomial) or in table form.

It should be noted that FIG. 1A illustrates schematically the layout of bladder 50 in a female, however the above procedure and description apply also to a male. The main difference between them is related to surrounding organs and does not have a substantial effect on the methods described herein, at least for some embodiments of the invention.

Signals and Signal Processing

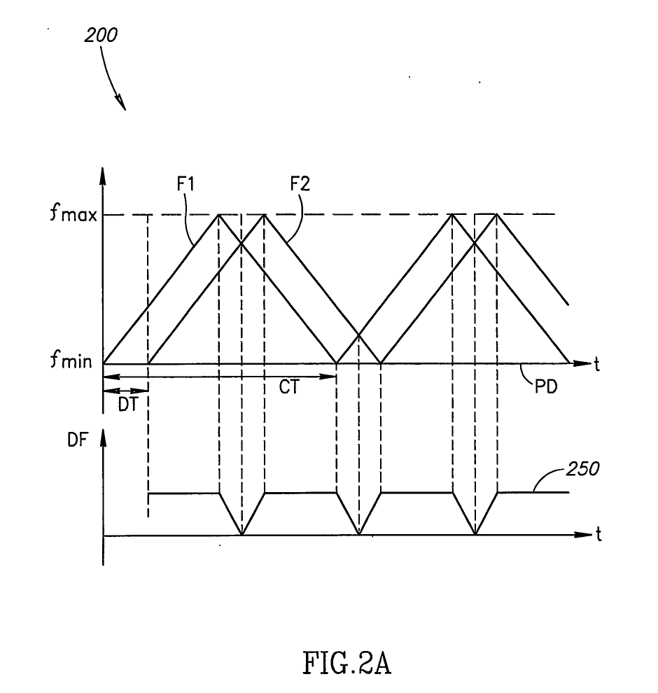

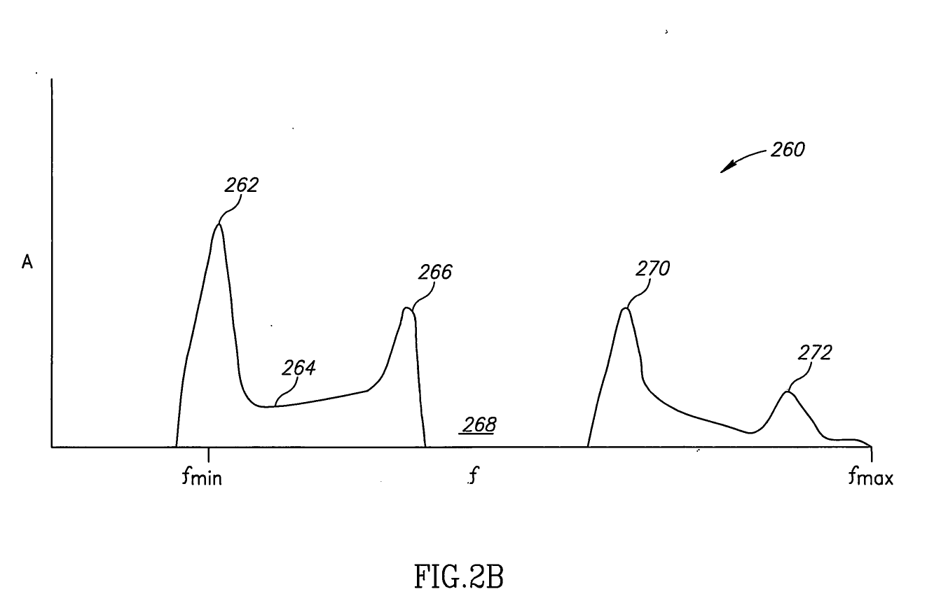

FIG. 2A is a graph 200 illustrating using a frequency sweeping method for calculating distance, according to an exemplary embodiment of the invention. FIG. 2A shows a transmitted signal F1 and one reflected (and received) signal F2 for a given reflector, such as the bladder front wall. The upper set of traces represent the variations of frequency with time and a lower trace 250 is the instantaneous difference in frequency between the transmitted and received signals. FIG. 2B shows a trace for multiple reflected signals and will be described subsequently.

In accordance with an exemplary embodiment of the invention, ultrasonic signal F1 is a frequency sweeping signal between a minimum frequency (fₘᵢₙ) and a maximum frequency (fₘₐₓ). This swept signal is transmitted into bladder 50. The first reflected signal of interest generally results from reflections from front bladder wall 60. As there is substantially no movement of the front bladder wall in a regular situation, F2 is a signal with the same cycle time CT and waveform as F1, and with no Doppler shift. However signal F2 will be detected with a time shift DT relative to signal F1, because the reflected signal travels twice the distance to the front wall as it travels to and fro.

The direct measurement of DT may not be practical and/or simple. However, subtracting F2 from F1 (in the frequency domain), results in a frequency shift (DF) dependent on the time delay. It should be noted that the frequency shift remains constant for nearly the entire duration of pulse F1 (if it is linear), thus providing more signal for detection. In an exemplary embodiment of the invention, the reflected signal F2 is optionally amplified (to normalize it with the transmitted signal) and subtracted (in the frequency domain) from the transmitted signal F1, and a frequency shift with a maximum value is found. Trace 250 shows the results of such a subtraction, which is a constant frequency (corresponding to a constant distance), except for changeover points at the ends of the sweep. In an exemplary embodiment of the invention, the subtraction of the frequencies is performed as heterodyning, optionally by using a same transducer for transmission and reception. Optionally, the detection is using an amplitude detector.

In an exemplary embodiment of the invention, transmitted signal F1 is transmitted with a pulse duration PD larger than a round trip propagation time of the signal through bladder 50 and back so that there is a one-to-one correspondence between frequency shift and distance. Optionally, PD is many times longer than the round-trip propagation time, so that received signals can be collected over a considerable time.

While a linear sweep is shown in FIG. 2, various sweeping functions may be used, in various embodiments of the invention. For example, symmetric or asymmetric, continuous or intermittent sweeps may be used. A potential advantage of an intermittent sweep is that far reflections are less likely to be swamped by near reflections. A continuous function may be used, for example, in an analog system. A potential advantage of symmetrical sweeping is that the relationship between transmitted and received signals stays constant (even if its polarity changes). A potential advantage of asymmetric sweeping is that the return sweep can be shortened. Another potential advantage is that interference time between F1 and F2 can be reduced. Optionally, the shape of the sweep function is linear (which provides a fixed frequency-time relationship and supports heterodyning in a single transceiver element), however other shapes, such as sinusoidal may be used. Optionally, processing during detection corrects for the effect of non-linear sweeping on the received frequencies. Optionally, the sweeping if not linear has a constant polarity of its derivative. Some sweep forms may result in simpler electronics and/or be more suited for the resonance responses of the acoustic unit. For example, both piezoelectric and PVDF materials are known to be more easily driven in certain frequencies and/or envelopes.

In FIG. 2A, a symmetrical saw tooth function is shown. While a sweep that is continuous and smooth over frequencies is shown, optionally, a stepped sweep is used, or the sweep may be comprised of discrete frequencies that the signal hops between. Optionally, periods of low amplitude are provided between hops.

In an exemplary embodiment of the invention, sweeping is used. However, TOF (time of flight) methods may be used to detect reflections from front and back walls of the bladder. Possibly the use of a frequency sweep function has the benefit of avoiding the need to use a damper since transmitted and received signals can be differentiated by their frequencies (at any given time). In an exemplary embodiment of the invention, TOF pulse based techniques use a correlation between a transmitted signal and its reflection to detect time of flight.

One processing method which may be used for sweeping based detection is subtracting the received signal (after amplification/normalization) from the transmitted signal, for example as part of signal detection, and detecting frequencies at which there are peaks in the amplitude of the subtracted signal, for example as part of signal processing. In some cases, the two largest peaks are the reflections from the front wall and from the back wall of the bladder. Optionally, a distance window is used to verify the identity of these signals. For example, signals that are (a) closer than a near bladder wall can be (or were in recent measurements) and/or (b) not at a distance about the distance recently found for the far wall, or (c) in a distance range for the far wall in general or for this patient, are ignored.

In an exemplary embodiment of the invention, a single transducer is used for transmitting and receiving. This transducer can automatically heterodyne the received signal with the transmitted signal, so that an AM detection circuit connected to the transducer receives a heterodyned signal (i.e., the difference signal). In an exemplary embodiment of the invention, the AM detecting circuit can be a relatively low frequency circuit, for example, 200 KHz, 100 KHz, or less.

In an exemplary embodiment of the invention, the sweep time is considerably longer than the longest time of interest (e.g., time to 150 mm and back, of the order of tens of microseconds), for example, up to 5, 10, 20, 50 times or more. In an exemplary embodiment of the invention, frequencies corresponding to a distance greater than the distance of interest are simply dropped out (e.g., not passed due to low frequency response) by the detector or by using a frequency filter. In an exemplary embodiment of the invention, the accumulation time (e.g., the time during which the heterodyned signal is meaningful) in a single sweep is 1 millisecond, 3 milliseconds, 5 milliseconds, 50 milliseconds, smaller, intermediate or greater values. For the frequencies discussed, the sweep time is also much longer than the cycle time of the frequency (e.g., 1 micro-second for 1 MHz).

In an exemplary embodiment of the invention, a calibration step (e.g., internal calibration) is used to convert sweep rates to times.

In an exemplary embodiment of the invention, the combination of low frequency and long accumulation times assists in using simpler processing circuitry and/or improving SNR, including ignoring noise resulting from reflections form other organs. Possibly, no digital circuitry is used.

In an exemplary embodiment of the invention, the instantaneous power used during sweeping is smaller than used for pulses, however, the total power used may be the same or greater. In an exemplary embodiment of the invention, using lower instantaneous power means that a lower voltage can be used to drive the transducer, for example, less than 15 volts. Optionally, the received signal is several millivolts, for example, more than one millivolt or more than 10 millivolts.

The sweeping rate (e.g., 1 KHz per mm, 0.5 KHz per mm, 2 KHz per mm, or smaller, intermediate or greater values) is optionally part of a tradeoff of a desired resolution in time and distance. Optionally, a distance resolution of 1 mm is enough.

Optionally, the continuous measurement is used to drop some measurements, for example, measurements which are more than 2 standard deviations (e.g., in amplitude or in location of peaks) than most other measurements. Optionally, only the most similar 90% of measurements are kept

FIG. 2B is a graph 260 showing a (simplified) received signal, after being converted to a frequency domain, in accordance with some embodiments of the invention. This signal is optionally generated by converting a received signal using an FFT or other method, optionally after subtracting the instantaneous transmission frequency (in the frequency domain, e.g., by subtracting the instantaneous frequency as a baseline/carrier frequency). The signal is optionally averaged or accumulated over time. A first peak 262 corresponds generally to the transmitted signal and reflections from the body surface. It is noted that in some embodiments of the invention, the transmitted signal is relatively wide, so that some received signals may be lower in frequency than fₘᵢₙ, Also, a wide signal may indicate detection of peaks by finding local maximums. A reflection 264 is from structures between the skin and the forward bladder wall. A reflection 266 is from forward bladder wall 60. A range of frequencies where there is no reflection, 268, indicates the lack of reflection from urine, which generally includes no interfaces and no dispersive elements. A reflection 270 is a reflection from back bladder wall 70. Often, reflection 270 is similar in size than reflection 266, or even larger, due to the focusing effect of the far bladder wall. Optionally, fill level is estimated based on a change in reflection from the far wall (or near wall), which is generally dependent on the distance (for the far wall) and the radius of curvature of the wall, even for a non-sweeped signal. In an exemplary embodiment of the invention, reflections 266 and 270 from the bladder walls are detected based on their contrast with no-reflection range 268. Optionally, typical patterns of echoic-to-unechoic and unechoic-to-echoic media are used to detect the wall(s). Alternatively or additionally, the signal level from the urine is used to calibrate a noise filter, which then blanks out the area of the urine. A reflection 272 may be a reflection form a rectum, cervix or uterus, however, is it optionally ignored due to its distance and ordinal position of reflection.

In an exemplary embodiment of the invention, one or more of the following techniques is used to improve signal to noise level, as well as or instead of other techniques of signal processing known in the art:

(a) active band pass filter, in which the detected signal is examined only at frequency bands corresponding to those where the walls (or other items of interest) were identified earlier;

(b) narrowband filter, in which processing on a wall surface or other item of interest is made more precise by filtering out frequencies other than those corresponding to the position of the wall;

(c) filtering on the driving signal, for example to better match it to or compensate for the transducer;

(d) filtering out of the transmitted signal or of reflections from the surface, optionally in a manner which prevents their amplitude from affecting any AGC; and

(e) noise threshold trigger level, which is optionally based on an expectation that there will be two strong peaks, so that signals significantly smaller than the two largest peaks can be damped out.

In a typical case, front wall 60 moves between 3 mm and 5 mm during bladder filling, at a distance of about 20 mm-50 mm from the skin, while back wall 70 moves from a distance (from the skin) of about 30 mm to a distance of about 10 mm. Optionally, measurements from the front wall are ignored, for example, as the movement of the back wall may be sufficient for an estimate of change in bladder filling and the approach of a time to empty the bladder. Alternatively or additionally, as change in front bladder wall position is slow, if a signal is not detected, a previous measurement is used.

In an exemplary embodiment of the invention, the walls are detected using frequency filters. Optionally, the settings of the filters are determined during calibration in which a range of distances are detected. In an exemplary embodiment of the invention, a near wall filter tapers off at frequencies corresponding to about 50 mm. If the near wall is not found in these 50 mm (for most people), there may be a contra-indicating physiological problem. If the far wall is also found in this filter, then the bladder is probably empty or near empty. A second, far wall filter, optionally covers the bandwidth corresponding to distances between 50 mm and 150 mm. Distances greater than 150 are optionally ignored as not being part of the bladder. Optionally, the various distances are found during a calibration step, where the filters are not fixed.

In an exemplary embodiment of the invention, missing measurements are extrapolated, for example, for short periods of time. Optionally, lack of measurement or a large change in a measurement, are used as indications of movement, so that temporally near measurements may be ignored.

In an exemplary embodiment of the invention, if motion is suspected a more intensive measurement mode is applied until it is confirmed what the suspected motion is. For example, the suspected motion may be confirmed as motion, as an intentional urination event or as enuresis.

Alternatively or additionally, a manual measurement may be suggested. In a manual measurement, the user is indicated to stand still (and optionally hold his/her breath) and press a switch when still, so that the device can operate without motion artifacts. Optionally, the user is requested to use a measuring cup and void, and then to check the volume voided.

FIG. 2B is schematic and does not show separate reflections for the (a) interface between the bladder wall and the surrounding tissue; and (b) interface between the bladder wall and urine. However, these are optionally detected as separate reflections, or possibly as notches on the side of the peaks caused by the walls. The impedance change (and this reflection amplitude) is generally larger for interface (b). Generally, the outside of the bladder is rough and the inside is smooth, so that reflection is more focused from the inside of the bladder. However, for the near bladder wall, the bladder wall acts as a dispersing lens. Thus, the reflection from the interface between tissue and front wall 60 may be significant as compared to that of the interface of front wall 60 with urine. For the rear wall, a converging effect is provided by the wall, so reflection is generally higher for the inner interface. Optionally, front wall 60 is used for detecting changes in wall thickness, for example those due to pressure changes or slow waves, or those due to Detruser's tension changes due to drug and/or artificial bladder control device (TENS).

In an exemplary embodiment of the invention, the transmitted beam is a converging beam designed to be reflected to be a near-parallel beam by front wall 60. Optionally, the beam is focused at a distance where bladder wall 60 is expected. Optionally, this focusing distance is found during a setup, where a plurality of focus lengths are tested and a distance where a signal from a front wall 60 s highest, is selected. Alternatively, the beams may be focused at a center of the viewed area (e.g., between bladder walls or where a far bladder wall will be at intermediate fill), or at a location where a back wall 70 is found at high fill levels (e.g., where it is far and accuracy may be important).

Optionally, signal processing includes non-sweeping analysis. In one example, once the distances to the two walls are found, these distances are used to create a time window for selectively receiving and processing signals only at times of arrival corresponding to the distances. Optionally, such analysis may include analyzing the texture of a bladder wall section based on reelection spectra from the wall. Alternatively or additionally, a more precise frequency sweeping is used to differentiate the two surfaces of a wall, by sending a short sweeping signal and analyzing reflections only at the time window.

Experiment

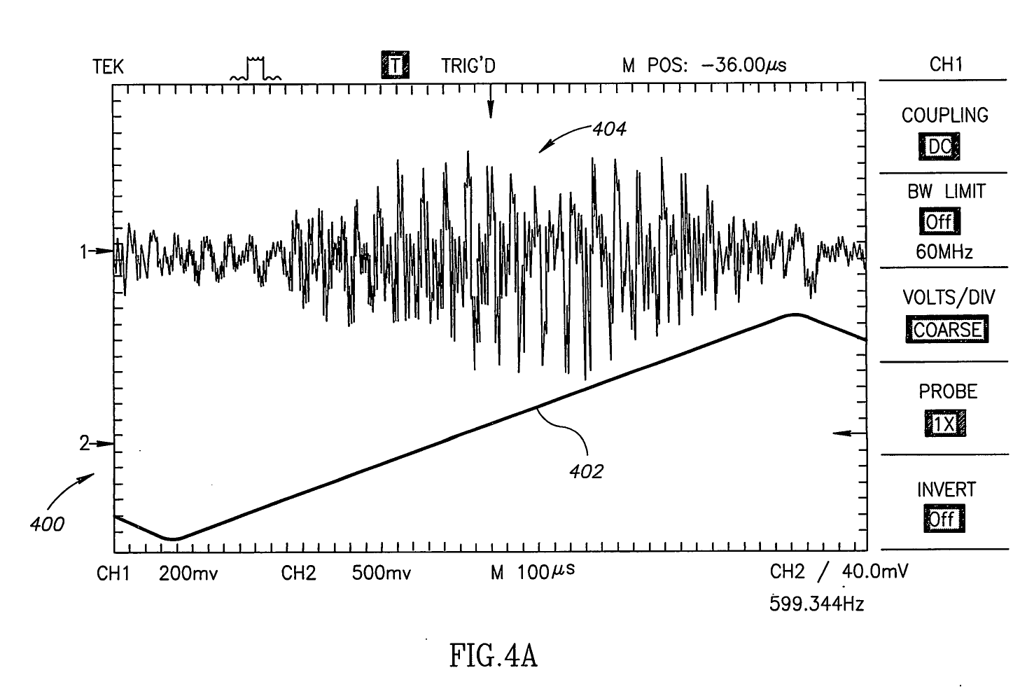

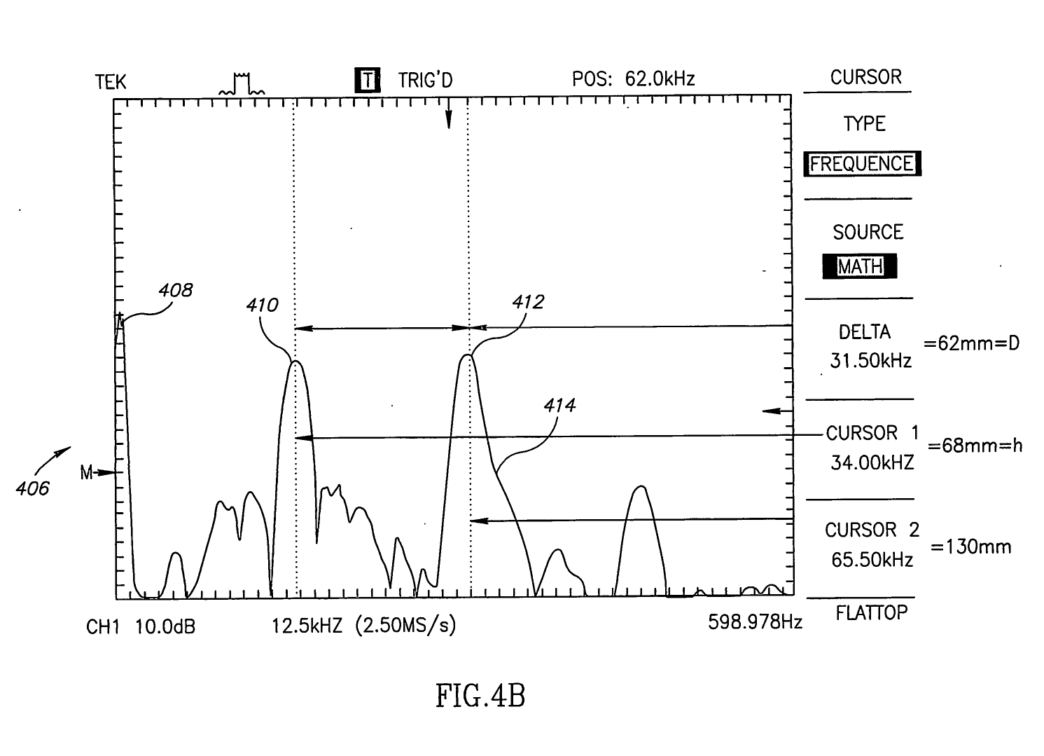

FIGS. 4A and 4B show the results of an experiment carried out on a phantom, a balloon of water. In FIG. 4A, a graph 400 shows a transmitted sweep 402 and a received signal 404.

In FIG. 4B, a graph 406 corresponds generally to FIG. 2B. A peak 408 from the surface/transmitter is shown. A peak 410 from the balloon wall is shown, a second peak 412 from the back wall of the balloon is also shown. The measurements of distances to and between the peaks correspond to the dimensions of the balloon. A notch/perturbation 414 in peak 412 is thought to be an effect of the wall thickness of the balloon.

Frequencies Used

Several considerations determine the frequencies used in exemplary embodiments of the invention. While it is noted that a wide range of frequencies may be used, some considerations may be relevant for some implementations and one or more of the following may be considered:

(a) The accuracy of the measured distance is limited by the wavelength used for measuring. The longer the wavelength the less accurate the resulting measurement.

(b) Absorption is higher for short wavelengths.

(c) Working in the far field may be desirable, this requires the frequency to be high enough for the area of interest (front wall 60) to be out of the near field.

(d) Focusing at short distances is easier for higher frequencies.

(e) The transmitter and receiver used (as well as the electronics) may be limited in frequency response.

(f) A desire to reduce cost and/or complexity.

(g) Coupling problems.

In an exemplary embodiment of the invention, typical frequencies used vary between 500 Khz to 1.6 Mhz, giving wavelengths of approximately 1-3 mm. More particularly, the range may be 800-2.5 KHz or 900-1500 KHz. Optionally, the range chosen between is greater, for example between 200 KHz and 4 MHz. Optionally, the sweep range is smaller for example, 200, 300, 400, 500, 600 or 700 KHz, or intermediate values. In an exemplary embodiment of the invention, the actual frequencies selected are not generally limited except by practical and/or anatomical considerations, and optionally by a desire that the combined geometry cause the area of interest to fall within a Fresnel area of the transducer. Various sweep rates may be used, for example, between 0.1 KHz/mm and 10 KHz/mm. However, the exact sweep rate selected may be a tradeoff between resolution and integration time and/or other parameters of device 30, such as detector dynamic range and depth of area of interest.

In one example, the following parameters are used for transmitting: a minimum sweep frequency (fₘᵢₙ) of 800 kHz, a maximum sweep frequency (fₘₐₓ) of 1200 kHz and a cycle time (CT) of 400 micro-seconds, so that the time-frequency relationship is 1 Hz/micro-second. Thus if DF is found to be 60 kHz, the propagation time of F2 traveling from the transmitter, to tissue and back is 60 micro-seconds, which means that the depth from which the signal was reflected is 60 micro-seconds/2×1500 m/sec=45 mm, with the division by 2 correcting for the fact the signal travels two ways. Optionally, acoustic velocity in soft tissue is measured rather than estimated, for example, using methods known in the art. Optionally, it is assumed that the velocities do not change significantly between people. Optionally, different estimated velocities are provided for different tissue types, for example, two or more of tissue surrounding bladder, bladder wall, fat and urine. Optionally, errors due to temperature changes are corrected, for example using a temperature sensor and correcting the tissue velocity based on the temperature. Optionally, however, a single value of 1540 m/s (an average value) is used for all tissues of interest.

In an exemplary embodiment of the invention, the transducer is a model K350/446 produced by the Keramos Corporation (USA), having a diameter of 24 mm (round), having a low Q factor of about 10-20 and an acoustic impedance of 15Mrayl. Smaller sizes (e.g., 10 mm) and other manufacturers can be used as well. Optionally, the shape (e.g., square) and/or other properties such as size of the transducer are selected to have a frequency resonance at about the center of operation, for example 1 MHz. In an exemplary embodiment of the invention, a lower Q is used to allow a wider range of frequencies for sweeping.

Device Design

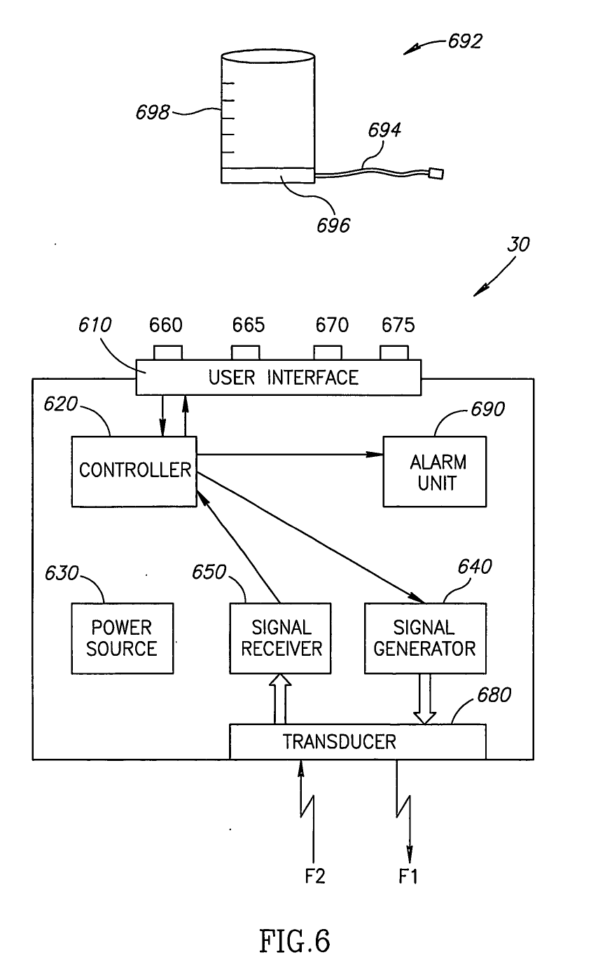

FIG. 6 is a schematic block diagram of bladder monitor device 30 in accordance with an exemplary embodiment of the invention. In an exemplary embodiment of the invention, bladder monitor device 30 comprises a controller 620 to control functionality of bladder monitor device 30. Optionally, controller 620 is connected to a signal generator 640 that prepares a frequency sweep function for transmission to a monitored person. Optionally, signal generator 640 is connected to a transducer 680, for example a piezoelectric transducer for transmitting an ultrasonic signal (F1) to the monitored person. In an exemplary embodiment of the invention, transducer 680 is additionally adapted to receive an ultrasonic signal (F2) reflected from the person responsive to the transmitted signal and transfer it to a signal receiver 650. Alternatively, separate transmitters and receivers may be used. Optionally, signal receiver 650 analyzes (e.g., filters and separates out) received signals and transfers the analysis results to controller 620. In some embodiments of the invention, controller 620 compares the transmitted signal and the received signals to determine a level of bladder distention and responds based on the determination. Alternatively, controller 620 transfers the signals to an external computer such as described below, for analysis.

Optionally, controller 620 and other circuitry therein are implemented as an ASIC. In an alternative embodiment, analog discrete processing circuitry is used.

In some embodiments of the invention, bladder monitor device 30 comprises a user interface 610 that includes an input interface and/or a display. Optionally, the user interface comprises an activation switch 660 for turning the device on or off. In some embodiments of the invention, user interface 610 comprises a selection dial 665 for selecting various modes of operation, for example including one or more of a calibration mode, a quiet mode and a load mode. Optionally, selection dial 665 selects a distention level for activating an alarm, for example that bladder monitor device 30 will initiate an alarm if the fill level exceeds 50%, 65%, 80% or 90%. In some embodiments of the invention, user interface 610 comprises a display 670, for example for providing status indications and/or to request actions from a user.

Optionally, switch 660 or another switch is used by a user to indicate urinary conditions, such as “bladder full, bladder empty and “urge to urinate felt”. Alternatively or additionally, the user interface can be used to enter indications such as fluid intake and body position. Other user interface designs can be used, for example a menu based design with one button to scan the menus and one button to enter values for a menu item.

In some embodiments of the invention, user interface 610 comprises a communication interface 675 (e.g. USB, serial, parallel, Bluetooth, wireless, IR, WiFi, cellular telephone unit), which may be used to connect the device to external devices, for example a personal computer for transferring measurement data to be analyzed. In another example, interface 675 is used for receiving instructions from an external device, for example to specify a range of frequencies to use for the transmitted signal and/or other parameters such as the sweep function. Optionally, device 30 acts as a web server. Optionally, device 30 can be plugged into a USB socket. Optionally, device 30 can be plugged into a cellular telephone, for example, for remote access to device 30 from a remote location. Optionally, a Bluetooth connection is used for such access. Optionally, interface 675 is used for transmitting an alert signal, for example to a caretaker. Optionally, device 30 communicates with a standard electronics equipment carried by the user, for example, a wireless headset, a cellular telephone, a pager, a PDA or a hearing device. Optionally, a dedicated wireless unit is provided, for example for alerting the user, with the wireless unit being worn as an earring or as a watch.

Optionally, interface 675 is used to connect to a separable processor (e.g., a PDA). Optionally, the processor is used to program device 30 and then the processor and user interface removed with only the alert and/or some minimal user interface forming part of device 30 that is worn all day. Optionally, the alert is also used to indicate to the user a desire to re-attach the user interface for more complex input. Optionally, device 30 is designed to be minimally visible, for example being thinner than 30 mm, 20 mm, 10 mm, or even thinner. Optionally, device 30 is not noticeable under everyday clothes. In an exemplary embodiment of the invention, device 30 weighs less than 200 gr, less than 100 gr or even less than 50 gr. Optionally, device 30 has a volume of less than 100 cc, 50 cc, less than 20 cc or even smaller.

Optionally, communication interface 675 is used for providing calibration information.

An automated measuring container 692 is shown, which includes a body 698, optionally graduated. A pressure sensor or other fill sensor 696 is optionally provided to generate a signal indicating a filling level of container 692. A cable 694 or wireless interface is optionally provided for automated feeding of the measured volume to device 30. Optionally, the feeding is in real-time, and at a high enough sampling (e.g., every second or faster), so that dynamic urination parameters, such as bladder emptying rate, can be tracked.

Alternatively or additionally, a measuring container as known in the art may be used and a resulting measure fed manually or automatically to device 30 or to a computer processing the stored values.

In some embodiments of the invention, bladder monitor device 30 comprises an alarm unit 690 to alert a user and/or caretaker of a situation which needs care, for example that bladder 50 has reached a pre-selected fill level and needs relief. Optionally, alarm unit 690 gives an audio indication, for example a siren. Alternatively or additionally, alarm unit 690 gives a visual indication, for example flashing lights. Alternatively or additionally, alarm unit 690 gives a tactile indication, for example tapping the user, causing vibrations or providing a small electric shock (possibly at a tingling level). Alternatively or additionally, alarm unit 690 communicates to an external device, for example by calling a caretaker (e.g. using a built in mobile phone or wireless transmitter) or by notifying an external device (e.g. a personal computer—not shown) to sound an alarm. Optionally, unit 690 generates a sound like a cellular telephone ring which is optionally programmable as known in the art of cellular telephones. Thus, when relief is required, the user can receive a “call” and pretend to leave a room in order to talk, rather than for urination.

In an exemplary embodiment of the invention, bladder monitor device 30 comprises a power source, for example a battery to supply power to enable the above-described functions. In an exemplary embodiment of the invention, 3 AAA batteries can provide sufficient power for several weeks or months of operation. Long operation can be provided, for example, if transducer 680 is activated only periodically, for example once every four minutes for one second (e.g., optionally providing multiple duty cycles in each second, for example, 10, 50 or 100 duty cycles). Other duty cycles, such as 0.5 seconds every 10 minutes or other number of minutes (e.g., such as 1, 3, 5, 7, 10) or other number of seconds (e.g., such as 0.1, 1, 4, 6) may be provided as well. Optionally, a higher sampling rate is (e.g., 10, 20, 50 or more samples a minute) used when the bladder fills, to make sure that a urination event is captured in high temporal resolution. A urination event as detected and/or measured by device 30 may be, for example, an intentional urination event or an unintentional event (e.g., caused by incontinence). Alternatively manual input is used. Optionally, power is saved by using a frequency window, so that once the approximate distance to a target (e.g., bladder wall) is known, the sweep range can be smaller (and have quite times between sweeps) and used in conjunction with a time window to selectively analyze reflections only from a relatively small range of distances.

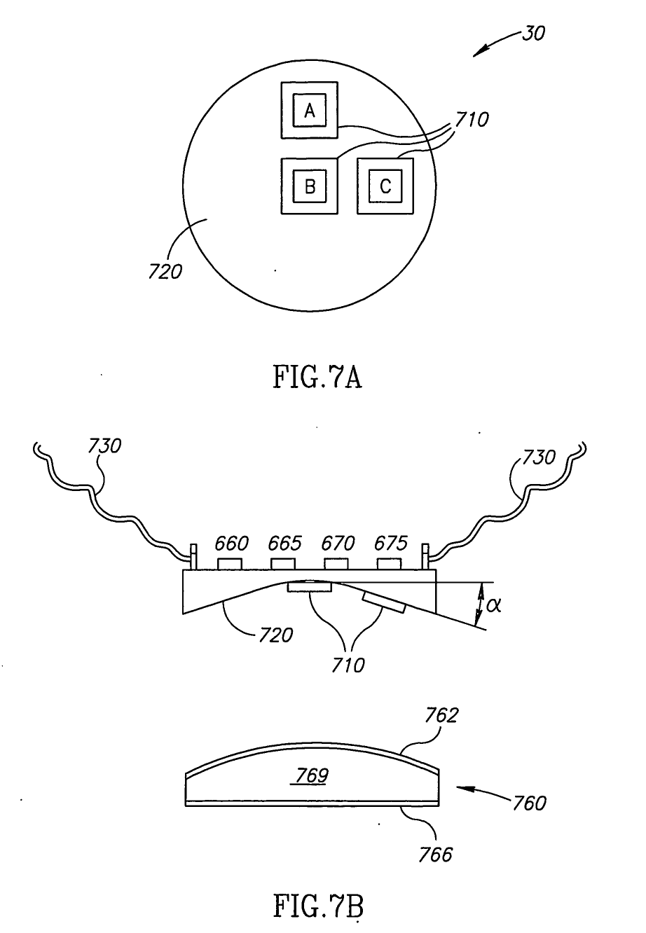

In an exemplary embodiment of the invention, device 30 is attached to the body using a strap 730 (FIG. 7B), optionally an elastic strap. In an exemplary embodiment of the invention, the strap is selected for one or more of comfort, lack of showing in clothing, and relative stability in location. Possibly, different shaped bodies and obesity levels will require different strap designs. Alternatively or additionally, an adhesive method is used, for example, tape or an adhesive layer of device 30. Gel or other acoustic coupling material may be used, for example as described below to provide a good acoustic path for the ultrasound. Optionally, attachment is at a relatively low contact pressure, to prevent distortion of the bladder by the pressure. Optionally, device 30 includes a pressure sensor which generates an alert if the contact pressure is too high.

In an exemplary embodiment of the invention, an adhesive pad 760 (FIG. 7B) is provided which is optionally designed to conform to a concave surface 720 (described below) of device 30 and/or to conform to an expected body curve of the user. In an exemplary embodiment of the invention, pad 760 comprises a gel coupling layer 769 with suitable acoustic properties. An optional adhesive layer 766 for attachment to the body is provided. Alternatively or additionally, an optional adhesive layer 762 for attachment to concave surface 720 is provided.

Transducer Layout

In an exemplary embodiment of the invention, transducer 680 is designed to improve the ease of use of device 30. In an exemplary embodiment of the invention, transducer 680 is arranged in a manner which is more likely to capture bladder 50. Optionally, multiple transducers are used and a best or composite signal (e.g., average) is used. Optionally, four, three, two or one transducers are used, to save power, rather than more transducers.

FIG. 7A is a schematic illustration of a transducer layout for bladder monitor device 30, in an exemplary embodiment including three transducers. FIG. 7B is a side cross-sectional view of bladder monitor device 30, in accordance with an exemplary embodiment of the invention.

In the embodiment shown, device 30 includes three transducers; however, two or more transducers can be used, for example, four transducers in a line.

In FIG. 7A, 3 transducers 710 (A, B and C) are shown. Optionally, transducer A is positioned highest on the user abdomen (transmitting with a slight downward tilt), transducer B is positioned according to the optimal position described herein and transducer C is positioned such that it transmits slightly from the right of the optimal position. In an exemplary embodiment of the invention, this arrangement covers a discovered off-center position of the bladder in some people and solves a difficulty in aiming of device 30. Optionally, other arrangements are used, for example, for people with left off-center bladders, left-positioned transducers may be used. In an exemplary embodiment of the invention, the arrangement used includes one or more transducers positioned along a line connecting the navel and genitals and at least one transducer off of this line. In an exemplary embodiment of the invention, an array of transducers is used and during calibration a transducer to be used (or an electrification scheme to emulate such a selection) is made. In informal experiments carried out by the inventor, 80% of the bladders are on the centerline. In 80%-90% of the people, the results on two channels (i.e., transducers) agree to within 1 mm. Optionally, the transducers are positioned so that a range of motion of the bladder may be captured. Optionally, a maximum value amount the transducers is accepted as a correct value.

In an exemplary embodiment of the invention, transducers 710 are positioned at different angles (e.g. a of 10 degrees as show in FIGS. 7A-7B) on a concave surface 720 to supply transmission signals at different angles toward bladder 50. In an exemplary embodiment of the invention, the concavity is selected based on the shape of the target body and/or to assist in correct aiming of the transducers. Optionally, surface 720 is a surface of a cone, not a cylinder. Alternatively, it may be a surface of a cylinder. Optionally, the transducer casings (the transducers are optionally flat) are designed so that placing thereof of surface 720 automatically aims them in a correct angle. Alternatively or additionally, suitably angled recesses may be formed in surface 720 to receive the transducers. Optionally, the distance between two 10 mm transducers is 0.5 mm. In an exemplary embodiment of the invention, the transducers are one above the other, along the line between navel and genitals, to compensate for accidental positioning of a transducer above pubic bone 80. One transducer is to the side, optionally to cover right-listing bladders.

Optionally, in use, transducers 710 transmit and receive signals simultaneously, optionally, using a different frequency sweep function or activated one after another. Optionally, the use of multiple transducers 710 allows selection of the clearest signal for each cycle in order to overcome effects of bladder movement, for example resulting from a user changing position or moving around. Additionally, multiple transducers 710 allow ignoring missing measurements from a single transducer. Optionally, different calibration values are stored for different transducers.

In some embodiments of the invention, after deployment, a single transducer 710 is selected with the clearest signal for future use.

Optionally, the beams from different transducers are designed to intersect in the middle of a full bladder. Alternatively, they may be designed to intersect at front wall 60 (to better receive dispersed reflections) or at back wall 70 in a full bladder situation (to better receive far-away signals). In some embodiments of the invention, the beam form takes into account the change in bladder radii (and thus dispersive and/or focusing effects). Optionally, the focusing of the transmitted and/or received beam is changed as the bladder fills and empties, for example using a controllable lens, for example, a water filled lens where changes in volume of water (or change in diameter) change the lens shape.

In an exemplary embodiment of the invention, an acoustic lens, optionally having a variable focal length, is used to control the focusing of the transmitted and/or received beams. Alternatively or additionally, different transducers have different focusing properties. In one example, one transducer is optimized for a front wall, one for a back wall in an intermediate fill situation and one for a back wall in a full fill situation.

In an exemplary embodiment of the invention, the transducer design is selected so that a beam from the transducer is converging where it hits near wall 60 and diverging where it hits far wall 70. In an exemplary embodiment of the invention, both walls are within a Fresnel zone of the transducer. In an exemplary embodiment of the invention, the transducer size, geometry and frequency are chosen for this effect. In one example, the formula “length=2*D²/wavelength” is used to calculate the start of the Fresnel zone (2 being a parameter, which may be experimentally determined and D being the dimension of the transducer). Optionally, the focusing and width of beam can be changed by varying element size and/or wavelength (e.g., also after manufacture). In an exemplary embodiment of the invention, a 110 mm square transducer is used with a 1 MHz central frequency.

As shown, each transducer is used for both transmitting and receiving. As noted above, this can be used for physical heterodyning of the transmitted and received signals, without any additional electronics. Optionally, separate transmitters and receivers are used. In one example, a relatively omni-directional receiver is used for multiple transmitters. Alternatively or additionally, a relatively omni-directional transmitter is used, for more focused receivers or for an omni-directional receiver. Optionally, the beam is between 10 degrees and 30 degrees in angular extent.

In some embodiments of the invention, an imaging sensor or an array sensor is used.

In some embodiments of the invention, acoustic gel such as used for ultrasound devices, is used to achieve optimal contact between a persons skin and bladder monitor device 30, for example by coating concave surface 720 with gel. In an exemplary embodiment of the invention, the gel is selected to maintain acoustic contact over a day or more, for example, 8, 24, 36 hours or more.

In an exemplary embodiment of the invention, transducer 710 produces an ultrasonic signal with a resonance between 500 Khz to 1500 Khz, for example 1 Mhz and a Q factor below 40 or even about or below 15.

Calibration

Different users have bladders with a different maximum size. Optionally, bladder monitor device 30 is calibrated to give fill values as a percentage of the maximum value for the specific user, or possibly as volume units (e.g., milliliters). Alternatively or additionally, device 30 is calibrated for acoustic properties. Optionally, calibration is performed by a health care provider. Alternatively, calibration is performed by a user or his caretaker, and is suitably automated and/or simplified.

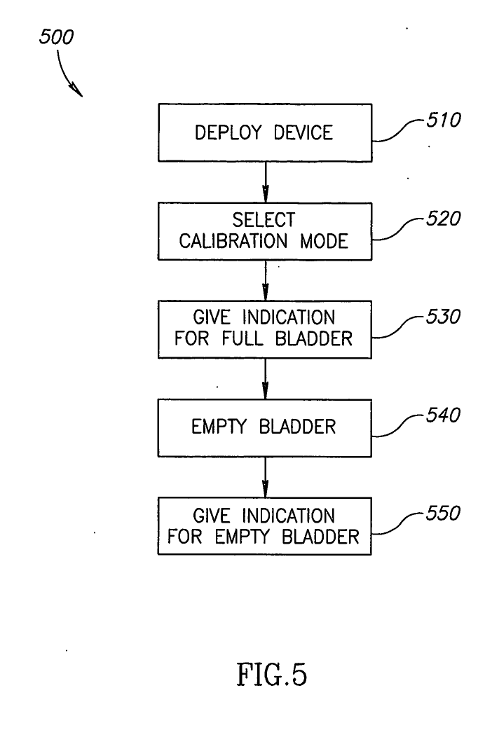

FIG. 5 is a flowchart 500 of a method of calibration, in accordance with an exemplary embodiment of the invention. Other calibration processes may be used, including processes in which some of the following acts are omitted.

At 510, bladder monitor device 30 is deployed. In an exemplary embodiment of the invention, a first act is determining a location where a suitable acoustic signal is detected. In an exemplary embodiment of the invention, a suitable acoustic signal is one which includes a reflection before and a reflection after a void area with no reflection (urine). Optionally, the deployment for calibration is on a bladder which is half full or completely full, to ensure a significant void area. Optionally, device 30 includes a LED to indicate a suitable signal is found.

In an exemplary embodiment of the invention, the location is marked with a tattoo, for example using a tattoo pen.

Some experimentation by the inventor has shown a suitable position (for a significant proportion of the adult population, if not all) to be along a line connecting the genitals and the navel. With the optimum location for some people being about 30 mm below a center of the line, typically about 30 mm above a pubic bone. In some people, an offset to the right of 0-15 mm is useful. In an exemplary embodiment of the invention, this offset is compensated for by the device design and not by moving the positioning of the device from the centerline. This location is generally about 125 mm below the navel. It is noted that many persons (even if modestly obese) have a flat surface on their abdomen at this location. This location will generally allow transmitting a signal to a most convex part of the bladder (where change is greatest during filling). It is noted that the best location in empty bladder situations may not be the same as for full bladder situations, as the bladder may move while filling and/or may fill non-uniformly (e.g., due to gravity). Optionally, a location that is best for a full bladder is selected. Alternatively or additionally, different transducers are optimally positioned for different fill levels.

Once device 30 is correctly positioned, fill level indications may be calibrated. In an exemplary embodiment of the invention, when the user feels a strong urge to urinate, the user selects calibration mode (520) on the device, for example by setting dial 665 to calibration mode. In some embodiments of the invention, setting to calibration mode indicates a full bladder. Alternatively, the user presses a switch, for example switch 660 (when in calibration mode) to indicate (530) that the bladder is full. The user then urinates to empty (540) the bladder. In some embodiments of the invention, bladder monitor device 30 automatically senses the empty bladder state as the minimum after recording a full bladder state. Alternatively, the user presses a switch, for example, pressing switch 660 twice to indicate (550) an empty bladder state.

In an exemplary embodiment of the invention, the measurements are used for calibrating a signal-to-fill level function. Optionally, the function is linear. Alternatively or additionally, a cubic or other estimation is used. Optionally, the function is stored in a table. Optionally, a more complex curve is used, for example a personal curve or one based on a population of people and the calibration is used to change the amplitude of the curve.

In some embodiments of the invention, user urinates into a measuring container (e.g., 692) and can determine an absolute volume by comparing the volume of urine with the change in distention measured by bladder monitor device 30. Optionally, the measuring container includes a sensor, for example a pressure sensor, which generates an indication of the volume of urine. Alternatively or additionally, a user input control is used, for example, each press indicating 10 ml.

It is noted that for some applications what may be of interest is a warning when urination should be performed. Thus, the actual volume may not be of interest, merely the fact that the bladder is full.

In some embodiments of the invention, bladder monitor device 30 automatically calibrates itself by noting maximum and minimum distensions over a set period of time, for example 1 hour, 12 hours, 24 hours, 36 hours or more. Optionally, when new maximum values are discovered, (or minimums where voiding is performed) recalibration may be performed.

In some embodiments of the invention, bladder monitor device 30 is originally deployed using an ultrasound scanner to determine an exact position of bladder 50 and deploy bladder monitor device 30 accordingly taking into account any abnormalities. Alternatively or additionally, a scanner may be used to detect a fill condition of the bladder.

In an exemplary embodiment of the invention, calibration values can be reset by turning off bladder monitor device 30 or by removing power source 630. Alternatively or additionally, bladder monitor device 30 may comprise a special switch or special memory erasing method, for example holding a switch in a certain position while turning on the device. In an exemplary embodiment of the invention, device 30 uses flash memory for storing tracked values and/or calibration values and/or processing results.

In an exemplary embodiment of the invention, intermediate fill levels are calibrated by collecting information over a plurality of urination events into a measuring cup. Optionally, a user is prompted to use the cup when a fill level that is different from ones previously calculated is detected and a user indicates a desire to urinate.

In an exemplary embodiment of the invention, proper calibration provides an accuracy in measuring distance of 0.5 mm over a distance of 0-150 mm. Lower accuracies, for example, 1 mm, 2 mm, 5 mm or worse may also be sufficient for many purposes. A higher accuracy, for example, 0.2 mm or 0.1 mm or better may also be provided. Optionally, fill level estimation is accurate to better than 20%, 10%, 5% or better, at least for higher fill levels over 60% or 70%. Optionally, volume estimation (or voiding volume) accuracy is better than 20%, 10%, 5% or better, at least for higher fill levels, even without personal calibration for volume.

If multiple sensors are provided, each sensor may be calibrated separately, or they may be calibrated simultaneously, albeit with different values.

Optionally, separate calibrations are performed for different body postures, for example standing and sitting, especially for low bladder volumes, for example 20%-40%. An article titled “MRI Assessment of the Influence of Body Position on the Shape and Position of the Urinary Bladder”, by Niels Kristian Kristiansen, Steffen Ringgaard, Hans Nygaard and Jens Christian Djurhuus, in Scand J Urol Nephrol 38: 53-61; 2004, DOI 10.1080/00365590310017325, the disclosure of which is incorporated herein by reference, suggests that there is little influence of body position for a full bladder.

Device Usage

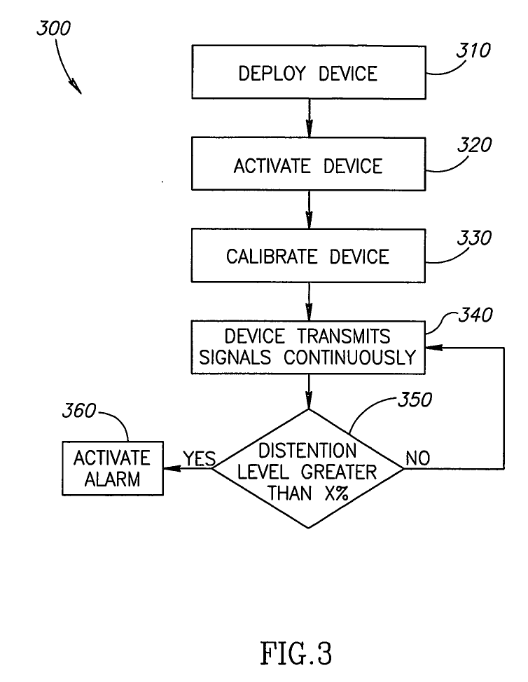

FIG. 3 is a flowchart 300 of a method of bladder filling monitoring in accordance with an exemplary embodiment of the invention.

In an exemplary embodiment of the invention, a user deploys (310) bladder monitor device 30. Often, the user is untrained. Optionally, the location for placing device 30 is marked with a tattoo pen or an indelible marker.

The user activates (320) the device by turning it on.

Optionally, the user performs calibration (330) based on the user's bladder 50, for example using the method described above. Optionally, calibration is performed in a body position of the user where the user would like to receive alerts, for example, standing, sitting or supine (e.g., for night time). Optionally, a user can switch between modes (which calibration to use) using the interface.

In an exemplary embodiment of the invention, bladder monitor device 30 continuously transmits signals (340) toward the user's bladder 50 and receives signals returned to the device. Bladder monitor device 30 analyzes the returned signals and determines a distention level of the user's bladder 50. Optionally, as noted above, the transmission is periodic, to conserve energy, and transmission rate may increase when fill levels approach fill levels where urination was previously performed. Optionally, a urination time is predicted based on fill level and fill rate.

If (350) the determined level is greater than a pre-selected fill percent (for example, 65%, 70%, 80% or 90%), bladder monitor device 30 activates (360) alarm unit 690 to notify the user or caretaker to take action, for example empty bladder 50.

Optionally, a user can query the device for an estimate of fill level. One example where such a query is useful is for training a user to recognize fill levels. By providing the user with an accurate feedback (that urination is not needed) a user may learn to overcome urge incontinence and/or an over-excited bladder.

In some cases device 30 is set to provide multiple alerts, for example as progressive threshold fill levels are detected.

Optionally, multiple parameters are used to evaluate need to urinate, including, for example fill level and one or more of wall thickness and fill rate.

In an exemplary embodiment of the invention, a user may remove and replace device 30 during a day, for example for the purpose of taking a shower. Optionally, device 30 requires recalibration. Alternatively or additionally, device 30 keeps track of the time that passed and assumes a constant fill rate over that time, for calibration purposes. Alternatively or additionally, a previous calibration is maintained. Alternatively or additionally, the transducers are used to detect if a same position of device 30 is maintained, for example, based on reflections from body structures other than bladder 50, for example the pubic bone. Optionally, a user voids before using device 30, for example prior to sleeping.

In an exemplary embodiment of the invention, device 30 is adapted for use with children, for example, being suitably sized, and is used as part of a diaper-training system or to help treat night wetting thereafter. Optionally, a loader alert is used for this application, as a person or child may need to be awakened.

In an exemplary embodiment of the invention, device 30 is used in hospitals or old-age homes to provide an indication to caretakers when a bed pan is needed and/or for preventing the replacement of diapers. Optionally, a plurality of devices 30 are connected to a single centralized alerting station manned by a caretaker.

In an exemplary embodiment of the invention, device 30 includes a clock and operates differently at different times of the day. In one example, different fill level thresholds for alerts are lower or higher at night. In another example, alert type (e.g., tactile or sound) or alert level are different at night. Optionally, an attention sensing method is used, by which, if a user did not respond (e.g., acknowledge by pressing a button) to a low alert level for a pre-threshold situation, a loader alert is used when a threshold is reached, as the user is assumed to be inattentive or asleep.

In an exemplary embodiment of the invention, device 30 is notified every time there is an incontinence event. Optionally, the threshold for alerts is reset to be below a fill level at which incontinence occurred. Optionally, a threshold level for alerting is determined by statistical analysis of incontinence levels. In one example, a threshold is determined by calculating a standard deviation and setting the threshold to be at two standard deviations from an average. Alternatively or additionally, a safety margin is added, for example a margin calculated as a percentage of fill, as an absolute number and/or based on a fill rate.

In an exemplary embodiment of the invention, device 30 is formed of low cost materials, so that it can be disposed of economically, for example after one day, one week or one or more months of use. In some embodiments of the invention, the low cost is due to the ability to use low frequency and low quality components, due to long integration time and low frequencies processed, as well as lack of specialized high voltage and pulse shaping circuitry. Optionally, the batter is non-replaceable, so once the battery is used up, the device is disposed of.

Experimental Results

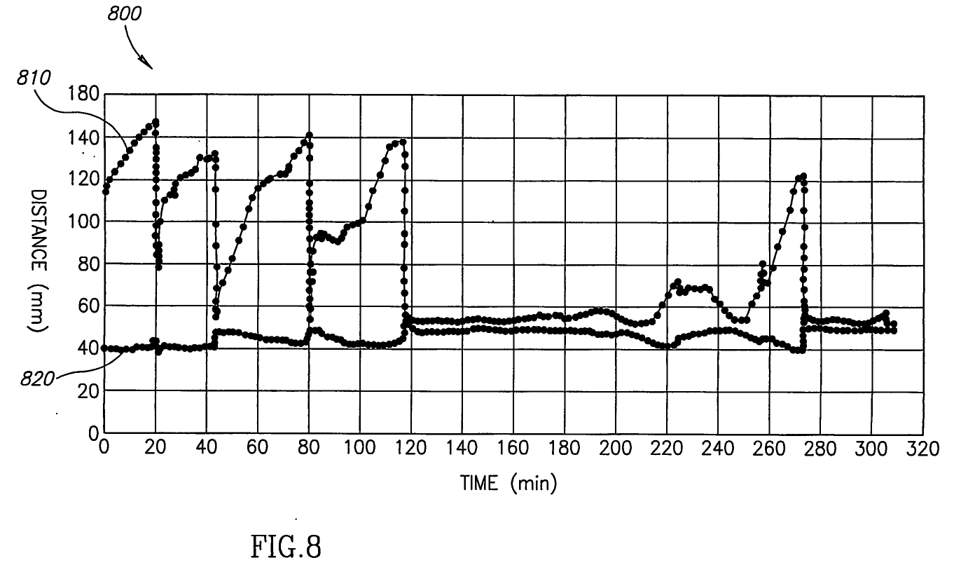

FIG. 8 is a graph 800 showing measurement of a bladder over 320 minutes in a real person. A line 810 is the distance (from device 30) to back wall 70 of the bladder and a line 820 is a distance to front wall 60. A plurality of filling and emptying events can be seen. In addition, a residual bladder fill level can be seen between emptying. Also visible are apparent undulations, these are possibly cased by bladder slow waves. In an exemplary embodiment of the invention, this is taken to be the detruser profile at an open sphincter situation which is affected by obstructions (peripheral resistance) and/or detruser activity. The variations in fill rate visible in graph 800 are caused by the person being told to drink large quantities of water over the first part of the period. In an exemplary embodiment of the invention, this is taken to be this is the profile of urine production by the kidneys. It is noted that at time 220, the person moved, causing a spike in the distance. However, it is noted that the concurrent motion of the near wall at least partially compensates for the error due to motion. Also, it is noted that the effects of such motion is generally more pronounced when the bladder is mostly empty. Optionally, device 30 is set to be more sensitive to motion artifacts when fill rates are low.

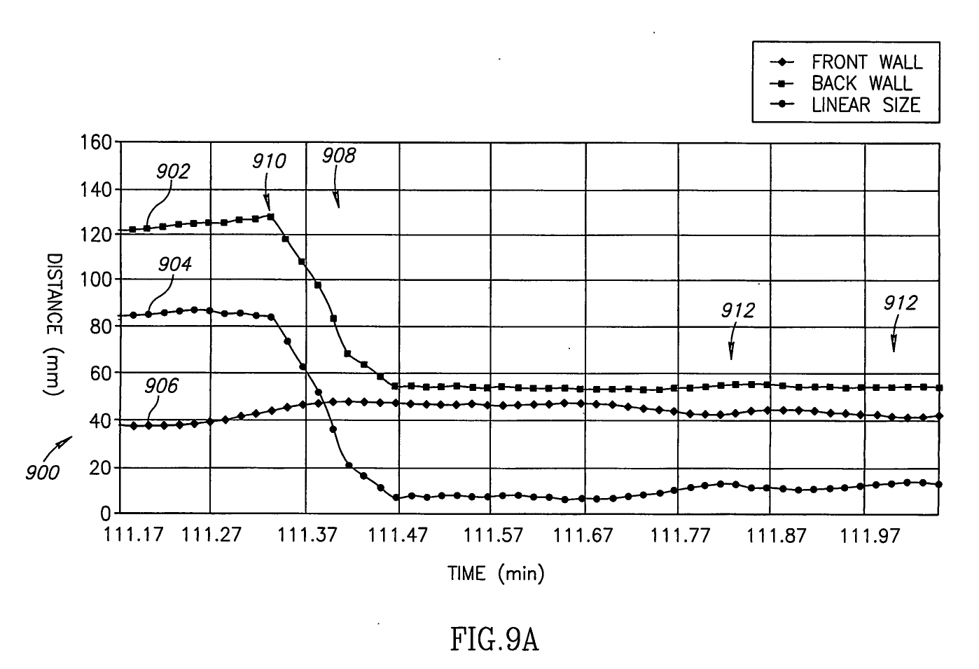

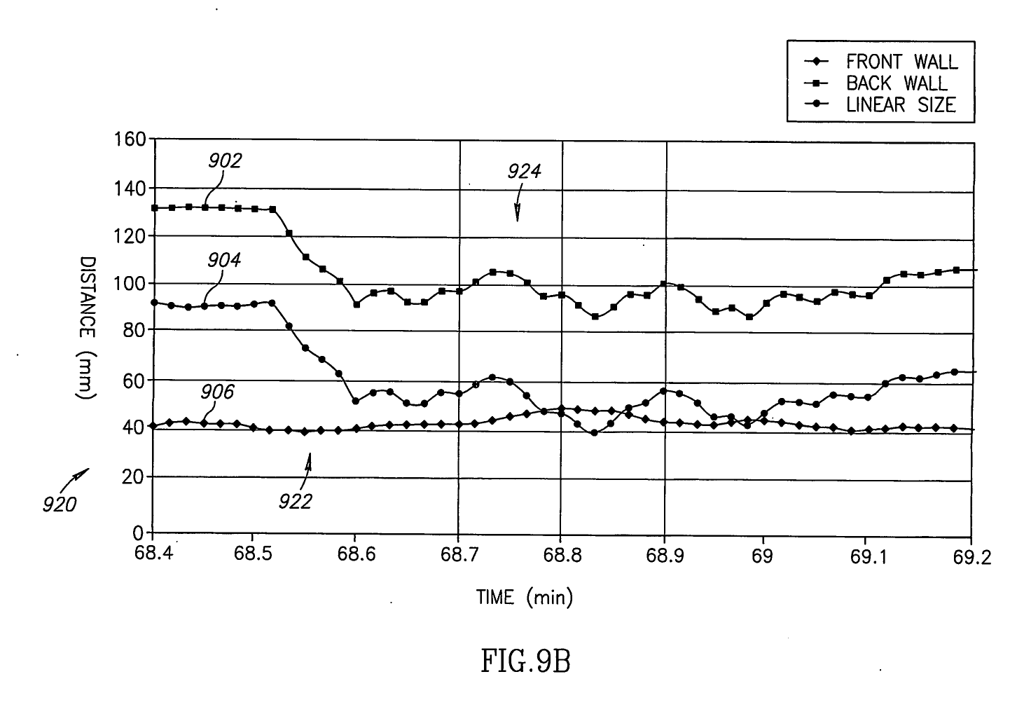

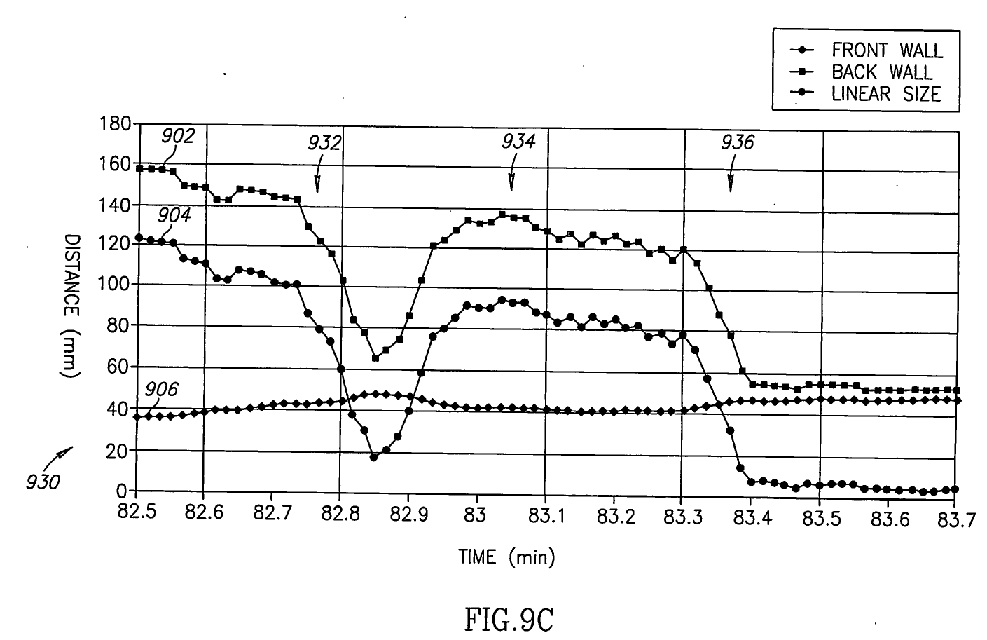

FIG. 9A is a graph 900 showing measurement results taken in real time during urination. A line 902 indicates the distance from device 30 to back bladder wall 70, a line 906 indicates the distance to forward bladder wall 60 and a line 904 indicates a distension (inter-wall distance), derived by subtracting line 906 from line 902. A reference 908 generally indicates a time of urination. Prior to this time, at a reference 910, a change in bladder position is observed just before and just as urination starts. Possibly, this indicates an increased intra-abdominal pressure preceding urination. A reference 912 indicates oscillations in the bladder geometry, possibly indicating post-urination muscle relaxation.

FIG. 9B is a graph 920 showing measurements associated with a partial urination event 922 in which a significant residual volume of urine is retained in the bladder, in the same person as in FIG. 9A. Lines 902-906 designate the same measurements as in FIG. 8. A series of oscillations 924 possibly indicate (forced) relaxation of the bladder as a result of interruption of the urination event. In an exemplary embodiment of the invention, this is taken to show detruser activity at a closed sphincter situation.

FIG. 9C is a graph 930 showing a two stage urination event. Lines 902-906 are as before. A first stage 932 shows a reduction in distension of the bladder, however, immediately after a recovery 934 appears to happen. It is hypothesized that this seeming recovery of urine volume is actually due to a change in shape of the bladder during stopping of urination while intra-abdominal muscles were still working. Optionally, measurements immediately after urination which is incomplete are ignored for this reason. Alternatively or additionally, pathological behavior of the person is detected by detecting such artifacts. A second stage 936 completes the emptying of the bladder.

In some experiments it was noted that changes in posture, such as sitting, standing and lying down affect the baseline of the measurement. However, returning to a previous posture causes the measurements to revert without a lasting effect. Also, as noted above, the effect is expected to be small.

Long Term Use

In an exemplary embodiment of the invention, device 30 is used for long term monitoring. In an exemplary embodiment of the invention, device 30 is used as a urination diary, by tracking urination events. Optionally, a user indicates input as well, for example, using a button every pressing of which indicates intake of one unit of liquid. Alternatively a more complex interface is used.

Optionally, urination data is periodically sent for analysis, for example by placing device 30 in a docking station attached to a computer or a telephone.

In an exemplary embodiment of the invention, long term monitoring and/or tracking is used for diagnosis, for example of prostate problems or of bladder control problems and/or to determine which drugs are effective in treating an existing problem.

Urodynamics

In an exemplary embodiment of the invention, device 30 is used for urodynamics. In a first use, device 30 is used to estimate a residual urine volume after urination. While the accuracy may be relatively poor, this value may still be of interest. Optionally, residual volume is calibrated using an imaging sensor or sensors to measure/estimate the volume, for low distension values. Alternatively or additionally, an extrapolation method is used, in which a correspondence between distension and volume change is worked back from the maximum fill level. Alternatively or additionally, a change in residual volume is tracked. It is noted that the position of maximum change in the bladder may change when the bladder is emptier. Optionally, the bladder position is tracked, for example using an imaging or an array sensor. Alternatively, different sensors are designated for different fill rates, with a calibration process optionally used to smooth the effect of transitioning between the sensors.

Optionally, the calibration for low fill levels also takes into account a model of changes in bladder volume, which model is optionally determined using imaging.

In an exemplary embodiment of the invention, measurement is made of the rate of emptying during a urination event, for example an event as shown in FIG. 9A. Optionally, such estimations are calibrated using a measuring device (e.g., 692) which provides real-time feedback during calibration on the rate of urine collection in the measuring device. Alternatively or additionally, bladder changes before, during and/or after urination are tracked. Alternatively or additionally, urine production rate is tracked.

In an exemplary embodiment of the invention, pressure levels in the bladder are determined and/or monitored. In an exemplary embodiment of the invention, bladder wall thickness or stiffness (e.g., acoustic velocity therein) is correlated with a pressure measured using other means, such as using a catheter. In some embodiments, tissue thickness and acoustic velocity may be inter-twined in a manner where by they cannot be separated. Possibly, thickness and velocity are separated by analyzing the rate of change and assuming different rates of change (e.g., frequencies) for each of thickness and velocity.

In an exemplary embodiment of the invention, device 30 is used to evaluate and/or modulate treatment protocols. In one example, the functioning of a stent or the efficacy of drugs on an enlarged prostate are tracked. Optionally, an input is provided on device 30 to indicate taking of drugs or other treatment. Alternatively or additionally, device 30 controls provision of drugs, for example increasing drug eluting or reducing it responsive to a desirable or undesirable effect on urination. Optionally, such controlled elution or other provision of drugs is used for other diseases as well, for example, to provide a diuretic action or to provide a drug that relaxes an over-excited bladder.

In an exemplary embodiment of the invention, a long term halter-like use of device 30 is to track bladder filling and emptying dynamics and/or flow problems, for example, for obstruction problems and/or for high residual volume problems.

Voiding Control

It is known in the art to provide persons in need thereof with a manual void control device, for example a catheter used for intermittent catheterization, a TENS device or a mechanical sphincter. Typically, a user of such a device uses a timer to decide when to activate a device. This may be wasteful and/or inconvenient. In an exemplary embodiment of the invention, a fill measuring device as described herein is used.

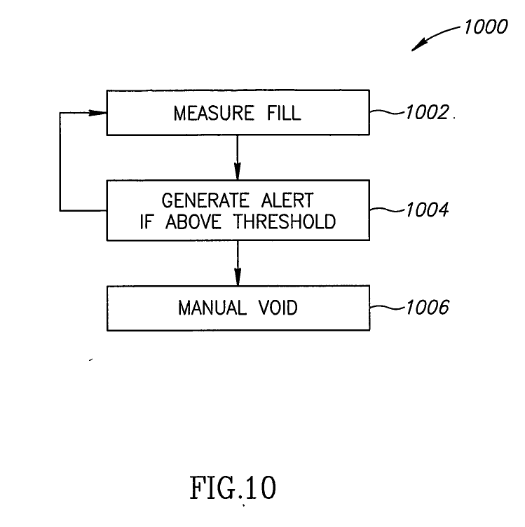

FIG. 10 is a flowchart 1000 of a void controlling method in accordance with an exemplary embodiment of the invention.

At 1002, a user measures a fill level. Optionally, device 30 is continuously worn. Alternatively, a hand-held device may be used, which is pressed to the abdomen when a fill indication is desired, for example based on a time schedule.

At 1004, if the fill level suggests voiding, an alert is optionally generated and the user can decide if to go and void.

At 1006, a user applies a manual voiding technique, for example intermittent catheterization.

Physiological Monitoring and Other Uses

In an exemplary embodiment of the invention, device 30 is used for uses other than preventing or assisting urination.

In an exemplary embodiment of the invention, device 30 is used for treating an over-excited bladder. In an exemplary embodiment of the invention, device 30 is used to detect slow-waves and track their changes. Optionally, slow waves are discovered based on changes in the wall thickness and/or wall position, optionally changes that are synchronized in time between the walls (possibly at a delay from each other). Alternatively or additionally, other bladder parameters are tracked, for example, one or more of bladder wall thickness, slow waves or pulsations with open or closed sphincter, emptying rate and/or emptying profile (e.g., to show obstruction information).

In an exemplary embodiment of the invention, oscillations in wall thickness and/or wall positions are tracked.

In an exemplary embodiment of the invention, a tracked parameter is compared to a template, for example a template derived from a same user or a different user or group of users. Different templates or different frequencies, number of oscillations and/or amplitudes may indicate diseased or normal conditions. Alternatively or additionally, diurnal and/or nocturnal functional capacity and/or activity are analyzed.

In an exemplary embodiment of the invention, a bio-feedback method is used, in which device 30 indicates to a user a frequency, amplitude and/or number of oscillations and a user uses the feedback to learn how to control them. Alternatively or additionally, the feedback is used to show a user that what seems to be a real need to urinate is really only an impulse, thereby teaching the use to control such impulses.

In an exemplary embodiment of the invention, device 30 is used for clinical evaluation, for example to determine effects or side effects of a drug, such as effect on fill rate or effect on feelings of fullness of bladder. In an exemplary embodiment of the invention, fill rate of the bladder indicates urine activity and may show the effects of diuretic and anti-diuretic pharmaceuticals or other treatments.

In an exemplary embodiment of the invention, device 30 is used for fluid management. In an exemplary embodiment of the invention, device 30 tracks fill rate and generates an alert if it is too low or too high (depending on the patient's condition). Optionally, device 30 provides instructions to a user, for example to partake of fluid or a diuretic or other drug. Optionally, the instructions are provided via an interface on device 30 or via an external device.

Variations and General

While the above description has focused on simple (e.g., with respect to imaging) devices, some embodiments of the invention are implemented using 2D or 3D imaging. Optionally, the 3D imaging ability is used sparingly (e.g., to save power or processing ability, especially when the user mobile), and the detection of reflections using frequency sweeping is used most of the time.

It will be appreciated that the above described methods and devices may be varied in many ways, including, changing the type of signal used. It should also be appreciated that the above described description of methods and apparatus are to be interpreted as including apparatus for carrying out the methods and methods of using the apparatus. Also included are various implementation methods, including hardware, software and firmware, in computers, in general purpose ultrasound machines or in dedicated ultrasound machines. Optionally, the software is provided on computer readable media, such as CR-ROMs, flash memory and diskettes. Also included within the scope of the invention are kits including one or more disposable devices and/or attachment pads, optionally sterilized or otherwise suitable for medical uses. For home use an instruction book is optionally provided.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art and are within the scope of some embodiments of the invention.

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Section headings are provided for navigation and should not be construed as necessarily limiting. The scope of the invention is limited only by the elements and limitations as used in the claims. When used in the following claims, the terms “comprise”, “include”, “have” and their conjugates mean “including but not limited to”.

1. A measuring device, comprising:
(a) an ultrasonic acoustic transceiver unit capable of sending and receiving acoustic signals into the body of a patient;
(b) frequency modulating circuitry which drives said transceiver unit with a signal whose frequency varies with time; and
(c) processing circuitry which extracts an indication of a distance, from at least one signal detected by said transceiver unit, said signal being a reflection of a transmission of said transceiver unit driven by said time varying frequency signal.

2. A device according to claim 1, wherein said signal is a sweeping frequency.

3. A device according to claim 2, wherein said sweeping is non-spatial.

4. A device according to claim 1, wherein said indication comprises an indication of a distance between a near wall and a far wall of a bladder.

5. A device according to claim 4, wherein said processing circuitry estimates a fill level of said bladder from said indication.

6. A device according to claim 5, comprising a memory having stored therein at least one calibration value used by said processing circuitry.

7. A device according to claim 6, including at least one user input control.

8. A device according to claim 7, wherein at least one of said at least one user input generates an indication that a bladder is full.

9. A device according to claim 7, wherein at least one of said at least one user input generates an indication that a bladder is just emptied.

10. A device according to claim 7, wherein at least one of said at least one user input is operative to receive a volume indication.

11. A device according to claim 1, wherein said processing circuitry is operative to automatically detect a maximum fill level condition by tracking fill level over time.

12. A device according to claim 5, wherein said processing circuitry is operative to estimate a residual fill volume.

13. A device according to claim 1, wherein said indication of a distance comprises a bladder wall thickness.

14. A device according to claim 5, wherein said processing circuitry is operative to estimate at least a bladder fill rate based on said indication.

15. A device according to claim 1, wherein said time varying frequency comprises a saw-tooth linear sweep.

16. A device according to claim 1, wherein said transceiver unit comprises a single ultrasonic element.

17. A device according to claim 1, wherein said transceiver unit generates a converging beam focused at a distance between 30 and 50 mm.

18. A device according to claim 1, wherein said transceiver unit generates a converging beam focused at a distance between 50 and 90 mm.

19. A device according to claim 1, wherein said transceiver unit generates a beam having a Fresnel zone covering a range of possible values for said distance.

20. A device according to claim 19, wherein said range is between 20 and 200 mm.

21. A device according to claim 1, wherein said transceiver unit comprises at least one transmitter and at least one separate receiver.

22. A device according to claim 1, wherein said transceiver unit comprises at least two transmitters and wherein said processing circuitry selects between the signal received from the two transmitters, for extracting said indication.

23. A device according to claim 22, wherein said two transmitters comprises at least three transmitters arranged two in a line and one off of the line, and each configured to aim a beam in a same general direction, but not parallel to each other.

24. A device according to claim 22, wherein said two transmitters transmit ultrasonic beams that are not parallel to each other.

25. A device according to claim 1, wherein said unit is mounted on a concave surface.

26. A device according to claim 1, comprising a strap of a length suitable for placing around a trunk of a human for mounting said unit adjacent a urinary bladder.

27. A device according to claim 1, comprising an adhesively removable gel pad adapted for coupling said transducer unit to human skin.

28. A device according to claim 1, comprising a memory storing a history of extracted indications.

29. A device according to claim 28, comprising a transmitter for transmission of data from said memory.

30. A device according to claim 28, wherein said memory serves as a urination diary memory.

31. A device according to claim 1, comprising an alert generator which generates an alert based on said indication.

32. A device according to claim 31, wherein said alert is tactile.

33. A device according to claim 1, wherein said acoustic transceiver unit transmits signals at between 200 KHz and 2000 KHz.

34. A device according to claim 1, wherein said time varying frequency varies from a minimum frequency to a maximum frequency over a time period greater than a travel time of signals at said frequency from a near wall to a far wall of a distended adult bladder.

35. A device according to claim 34, wherein said time period is at least 10 times the travel time.

36. A device according to claim 34, wherein said time period is at least 20 times the travel time.

37. A device according to claim 1, wherein said processing circuitry is adapted for processing only frequencies lower than 200 KHz.

38. A device according to claim 1, wherein said processing circuitry is adapted for processing only frequencies lower than 150 KHz.

39. A device according to claim 1, wherein said transceiver comprises a piezoelectric material excited with less than 13V.

40. A device according to claim 1, wherein said transceiver comprises a piezoelectric material excited with less than 5V.

41. A device according to claim 1, wherein said transceiver comprises at least one transducer which acts as both a transmitter and a receiver and which heterodynes a received signal with a transmitted signal.

42. A device according to claim 1, comprising a frequency filter which drops frequencies corresponding to distances not of interest.

43. A device according to claim 1, wherein said processing circuitry accumulate the contribution of at least 1 millisecond of received signals for said extracting.

44. A method of measuring a parameter of a bladder, comprising:
(a) transmitting a time-varying frequency modulated ultrasonic signal at a bladder;
(b) receiving a reflection of said signal from at least a portion of said bladder; and
(c) extracting at least an indication of a distance from a frequency of said reflection.

45. A method according to claim 44, wherein transmitting a time-varying signal comprises transmitting a plurality of time-varying signals from spatially separated transducers.

46. A method according to claim 45, comprising selecting a best reflection.

47. A method according to claim 44, wherein said indication comprises a distance to a far wall of said bladder.

48. A method according to claim 44, wherein said indication comprises a wall thickness.

49. A method according to claim 44, wherein said indication comprises a distance between a near wall and a far wall of said bladder.

50. A method according to claim 49, comprising converting said indication into an estimation of a fill level of said bladder.

51. A method according to claim 50, wherein said fill level comprises a fill volume.

52. A method according to claim 50, comprising generating an alert to a user responsive to said fill level.

53. A method according to claim 50, comprising calibrating said indication to a fill level.

54. A method according to claim 53, wherein calibrating comprises receiving a filling indicator from a user.

55. A method according to claim 54, comprising measuring an output of urine in a measuring device outside the body.

56. A method according to claim 55, wherein said device generates an electronic signal reflecting a urine volume.

57. A method according to claim 53, wherein calibrating comprises automatically tracking filling and emptying behavior of said bladder.

58. A method according to claim 44, wherein said indication comprises a thickness of a bladder wall.

59. A method according to claim 58, comprising extracting an indication of bladder slow waves in said bladder.

60. A method according to claim 44, comprising extracting a bladder fill rate from said indication.

61. A method according to claim 44, comprising extracting a urination rate from said indication.

62. A method according to claim 44, comprising extracting a residual urine volume from said indication.

63. A method according to claim 44, comprising tracking and storing said indication over time.

64. A method of calibrating a bladder urine fill detector device, comprising:
(a) measuring a volume of urine from a urination event;
(b) measuring a physical parameter of a bladder in association with said event; and
(c) storing a correspondence between said volume and said physical parameter in said detector device.

65. A method according to claim 64, wherein measuring comprises measuring a distension of said bladder using a frequency sweeping method.

66. A method according to claim 64, wherein said parameter comprises a volume of urine.

67. A method according to claim 66, wherein said volume is measured using a sensor and automatically provided to said detector device.

68. A method according to claim 64, wherein said parameter comprises a bladder wall thickness.

69. A method of measuring bladder distension, comprising:
(a) sending a measurement signal to an off-center bladder to measure a one dimensional geometrical parameter of said bladder; and
(b) estimating a distension of said bladder from said measured parameter.

70. A method of detecting a wall of a bladder, comprising:
(a) detecting a level of acoustic signal ostensibly from urine; and
(b) searching for a signal from a bladder wall to be greater in amplitude than the detected level.

71. A method of estimating a bladder fill level above 70% thereof, comprising:
(a) transmitting an acoustic signal to the bladder;
(b) determining an indication of a distension of said bladder in one dimension; and
(c) determining a fill level of said bladder to be above 70%, from said indication, using at least one personalized calibration value.

72. A method according to claim 71, wherein said acoustic signal comprises a frequency swept scalar signal.

73. A method of selecting device parameters, comprising:
(a) providing a device design for use in a body geometry and having at least one of a selectable acoustic transducer geometry and an operating frequency; and
(b) selecting at least one of said operating frequency and said body geometry such that said body geometry lies within a Fresnel zone of said transducer operating at said frequency.
